(19) United States
(12) Patent Application Publication
(10) Pub. No.: US 2023/0149415 A1
(43) Pub. Date: May 18, 2023

(54) METHODS AND COMPOSITIONS FOR TREATING CANCER

(71) Applicant: Engine Biosciences Pte. Ltd., Singapore (SG)

(72) Inventors: Stephen Harrison, El Granada, CA (US); Christine Taylor Brew, Montara, CA (US); Michael David Winther, Singapore (SG); Sourabh Banerjee, Singapore (SG); Shawn Yost, San Mateo, CA (US)

(21) Appl. No.: 17/936,089

(22) Filed: Sep. 28, 2022

Related U.S. Application Data
(63) Continuation of application No. PCT/US2021/025230, filed on Mar. 31, 2021.
(60) Provisional application No. 63/003,736, filed on Apr. 1, 2020.

(51) Int. Cl. A61K 31/5377; A61K 45/06; A61K 31/519

(52) U.S. Cl. CPC A61K 31/5377; A61K 45/06; A61K 31/519

(57) ABSTRACT

Methods and compositions for treating cancer are disclosed herein. The methods may comprise use of therapeutically effective amounts of one or more therapeutic agents to cause a difference in expression or activity of protein kinase, membrane associated tyrosine/threonine 1 (PKMYT1) in cancer cells that are deficient in protein phosphatase 2 regulatory subunit B alpha (PPP2R2A).

### CROSS-REFERENCE

This application is a continuation application of International Application No. PCT/US2021/025230, filed Mar. 31, 2021, which application claims the benefit of U.S. Provisional Application No. 63/003,736, filed Apr. 1, 2020, all of which applications are incorporated herein by reference.

### BACKGROUND

Despite advances, treatment of cancer (e.g., liver or ovarian cancer) remains relatively difficult. Systemic treatments such as chemotherapies may be toxic and may have negative side effects on patients. Moreover, the lack of specific biomarkers can complicate development or use of targeted treatments.

One approach for treating cancer cells includes identifying target genes and biomarkers which identify which cancer cells may be sensitive to alteration in the activity of those target genes.

### SUMMARY

As recognized herein, identifying synthetic lethal gene pairs, in which an inhibition of both genes or inhibition or one gene in the presence of a mutation or deletion in a second gene may lead to cell death, may be useful therapeutically in killing cancer cells while maintaining viability of non-cancer cells.

Recognized herein is a need for therapeutic agents for targeted treatments and therapies for treating a subject having or suspected of having a cancer (e.g., liver or ovarian cancer). The subject having or suspected of having a cancer may have, in one or more cancer cells of the cancer, a mutation in, deletion in, difference in (e.g., a decrease or increase in) expression of, or difference (e.g. decrease, increase or alteration) in activity level of a first gene compared to a healthy or non-cancer control. Disclosed herein are methods and compositions for treating a cancer or cancer cell or tissue having such a difference in (e.g., a decrease or increase in) expression or a difference (e.g. decrease, increase or alteration) in activity level of the first gene by causing a difference in (e.g., a decrease or increase in) expression or activity of a second gene in the cancer or cancer cell, thereby treating the subject. The first gene and the second gene may form a synthetic lethal pair. The first gene may encode a regulatory protein (e.g. that regulates the cell cycle), and the second gene may encode a therapeutically modifiable protein (e.g. a kinase).

In an aspect, disclosed herein is a method for treating a subject having or suspected of having a cancer (e.g., liver or ovarian cancer), comprising administering to the subject a therapeutically effective amount of one or more therapeutic agents that causes a difference in (e.g., a decrease or increase in) expression or activity of protein kinase, membrane associated tyrosine/threonine 1 (PKMYT1) or WEE1 G2 checkpoint kinase (WEE1) in the subject, thereby treating the subject for the cancer, wherein the cancer is associated with cancerous tissue comprising a cell that has a difference in expression or activity level of Protein Phosphatase 2 (PP2A) or a subunit thereof as compared to a healthy control, or wherein the cancer is associated with cancerous tissue comprising a cell that displays mutations and/or deletions in genes encoding subunits of Protein Phosphatase 2 (PP2A) as compared to a healthy control.

In some embodiments, the cancer is associated with cancerous tissue comprising a cell that has a difference in expression or activity level of Protein Phosphatase 2 (PP2A) or a subunit thereof as compared to a healthy control.

In some embodiments, the cancer is associated with cancerous tissue comprising a cell that displays mutations and/or deletions in genes encoding subunits of Protein Phosphatase 2 (PP2A) as compared to a healthy control. In some embodiments, the presence or absence of the mutations and/or deletions is identified by an assay of cells derived from tissue obtained from the subject. In some embodiments, the assay is a next generation sequencing-based assay.

In some embodiments, the one or more therapeutic agents comprise one or more members selected from the group consisting of a small molecule (e.g., a molecule having a molecular weight of less than 900 Daltons), a protein, an intrabody, a peptide, a ribonucleic acid (RNA) molecule, and, an endonuclease complex and a deoxyribonucleic acid (DNA) construct.

In some embodiments, the DNA construct comprises an endonuclease gene. In some embodiments, the endonuclease gene encodes a CRISPR associated (Cas) protein. In some embodiments, the Cas is Cas9.

In some embodiments, the DNA construct comprises a guide RNA targeting a PKMYT1 gene.

In some embodiments, the endonuclease complex comprises an endonuclease. In some embodiments, the endonuclease is a CRISPR associated (Cas) protein.

In some embodiments, the small molecule comprises a PKMYT1 inhibitor. In some embodiments, the PKMYT1 inhibitor comprises 5-((5-methoxy-2-((4-morpholinophenyl)amino)pyrimidin-4-yl)amino)-2-methylphenol, N-(2-chloro-6-methylphenyl) ((6-(4-(2-hydroxyethyl)piperazin-1-yl)-2-methylpyrimidin-4-yl)amino)thiazole-5-carboxamide (dasatinib), 4-((2,4-dichloro-5-methoxyphenyl)amino)-6-methoxy-7-(3-(4-methylpiperazin-1-yl)propoxy)quinoline-3-carbonitrile (bosutinib), N-(5-chlorobenzo[d][1,3]dioxol-4-yl)-7-(2-(4-methylpiperazin-1-yl)ethoxy)-5-((tetrahydro-2H-pyran-4-yl)oxy)quinazolin-4-amine (saracatinib), (E)-N-(4-((3-chloro-4-fluorophenyl)amino)-3-cyano-7-ethoxyquinolin-6-yl)-4-(dimethylamino)but-2-enamide (pelitinib), N-(3-chlorophenyl)-6,7-dimethoxyquinazolin-4-amine (tyrphostin AG 1478), 6-(2,6-dichlorophenyl)-2-((4-(2-(diethylamino)ethoxy)phenyl)amino)-8-methylpyrido[2,3-d]pyrimidin-7(8H)-one (PD-0166285), 6-(2,6-dichlorophenyl)-8-methyl-2-((4-morpholinophenyl)amino)pyrido[2,3-d]pyrimidin-7(8H)-one (PD-173952), 6-(2,6-dichlorophenyl)-8-methyl-2-((3-(methylthio)phenyl)amino)pyrido[2,3-d]pyrimidin-7(8H)-one (PD-173955), or 6-(2,6-dichlorophenyl)-2-((4-fluoro-3-methylphenyl)amino)-8-methylpyrido[2,3-d]pyrimidin-7(8H)-one (PD-180970).

In some embodiments, the small molecule comprises a WEE1 inhibitor. In some embodiments, the WEE1 inhibitor comprises 6-(2,6-dichlorophenyl)-2-((4-(2-(diethylamino)ethoxy)phenyl)amino)-8-methylpyrido[2,3-d]pyrimidin-7(8H)-one (PD-0166285), 2-allyl-1-(6-(2-hydroxypropan-2-yl)pyridin-2-yl)-6-((4-(4-methylpiperazin-1-yl)phenyl)amino)-1,2-dihydro-3H-pyrazolo[3,4-d]pyrimidin-3-one (MK-1775), 9-hydroxy-4-phenylpyrrolo[3,4-c]carbazole-1,3(2H,6H)-dione (PD-407824), 6-butyl-4-(2-chlorophenyl)-9-hydroxypyrrolo[3,4-c]carbazole-1,3(2H,6H)-dione, or 6-(2-chloro-6-fluorophenyl)-2-((2,4,4-trimethyl-1,2,3,4-tetrahydroisoquinolin-7-yl)amino)imidazo[1,2-a]pyrimido[5,4-e]pyrimidin-5(6H)-one.

In some embodiments, the PP2A subunit is selected from the group consisting of 65 kDa regulatory subunit A alpha (PPP2R1A), 65 kDa regulatory subunit A beta (PPP2R1B), 55 kDa regulatory subunit B alpha (PPP2R2A), 55 kDa regulatory subunit B beta (PPP2R2B), 55 kDa regulatory subunit B gamma (PPP2R2C), 55 kDa regulatory subunit B delta (PPP2R2D), 72/130 kDa regulatory subunit B (PPP2R3A), 48 kDa regulatory subunit B (PPP2R3B), regulatory subunit B″ subunit gamma (PPP2R3C), regulatory subunit B′ (PPP2R4), 56 kDa regulatory subunit alpha (PPP2R5A), 56 kDa regulatory subunit beta (PPP2R5B), 56 kDa regulatory subunit gamma (PPP2R5C), 56 kDa regulatory subunit delta (PPP2R5D), 56 kDa regulatory subunit epsilon (PPP2R5E), catalytic subunit alpha (PPP2CA), and catalytic subunit beta (PPP2CB). In some embodiments, the PP2A subunit is PPP2R2A.

In some embodiments, the method disclosed herein further comprises administering to the subject a therapeutically effective amount of one or more therapeutic agents that causes a difference in (e.g., a decrease in) expression or activity of PPP2R2A.

In some embodiments, the healthy control is from one or more subjects that do not exhibit the cancer (e.g., liver or ovarian cancer).

In some embodiments, the cancerous tissue is breast tissue, pancreatic tissue, uterine tissue, bladder tissue, colorectal tissue, prostate tissue, liver tissue, or ovarian tissue. In some embodiments, the cancerous tissue is liver tissue. In some embodiments, the cancerous tissue is ovarian tissue.

In another aspect, disclosed herein is a composition for treating a subject having or suspected of having a cancer (e.g., liver or ovarian cancer), comprising a formulation comprising at least one therapeutic agent, wherein the at least one therapeutic agent is present in an amount that is effective to cause a difference in (e.g., a decrease or increase in) expression or activity of protein kinase, membrane associated tyrosine/threonine 1 (PKMYT1) or WEE1 G2 checkpoint kinase (WEE1) following administration to the subject, and wherein the cancer is associated with cancerous tissue comprising a cell that has a difference in expression or activity level of Protein Phosphatase 2 (PP2A) or a subunit thereof as compared to a healthy control, or wherein the cancer is associated with cancerous tissue comprising a cell that displays mutations and/or deletions in genes encoding subunits of Protein Phosphatase 2 (PP2A) as compared to a healthy control.

In some embodiments, the cancer is associated with cancerous tissue comprising a cell that has a difference in expression or activity level of Protein Phosphatase 2 (PP2A) or a subunit thereof as compared to a healthy control.

In some embodiments, the cancer is associated with cancerous tissue comprising a cell that displays mutations and/or deletions in genes encoding subunits of Protein Phosphatase 2 (PP2A) as compared to a healthy control. In some embodiments, the presence or absence of the mutations and/or deletions is identified by an assay of cells derived from tissue obtained from the subject. In some embodiments, the assay is a next generation sequencing-based assay.

In some embodiments, the at least one therapeutic agent comprises one or more members selected from the group consisting of a small molecule (e.g., a molecule having a molecular weight of less than 900 Daltons), a protein, an intrabody, a peptide, a ribonucleic acid (RNA) molecule, and, an endonuclease complex and a deoxyribonucleic acid (DNA) construct.

In some embodiments, the DNA construct comprises an endonuclease gene. In some embodiments, the endonuclease gene encodes a CRISPR associated (Cas) protein. In some embodiments, the Cas is Cas9.

In some embodiments, the DNA construct comprises a guide RNA directed to a PKMYT1gene.

In some embodiments, the endonuclease complex comprises an endonuclease. In some embodiments, the endonuclease is a CRISPR associated (Cas) protein.

In some embodiments, the small molecule comprises a PKMYT1 inhibitor. In some embodiments, the PKMYT1 inhibitor comprises 5-((5-methoxy-2-((4-morpholinophenyl)amino)pyrimidin-4-yl)amino)-2-methylphenol, N-(2-chloro-6-methylphenyl)-2-((6-(4-(2-hydroxyethyl)piperazin-1-yl)-2-methylpyrimidin-4-yl)amino)thiazole-5-carboxamide (dasatinib), 4-((2,4-dichloro-5-methoxyphenyl)amino)-6-methoxy-7-(3-(4-methylpiperazin-1-yl)propoxy)quinoline-3-carbonitrile (bosutinib), N-(5-chlorobenzo[d][1,3]dioxol-4-yl)-7-(2-(4-methylpiperazin-1-yl)ethoxy)-5-((tetrahydro-2H-pyran-4-yl)oxy)quinazolin-4-amine (saracatinib), (E)-N-(4-((3-chloro-4-fluorophenyl)amino)-3-cyano-7-ethoxyquinolin-6-yl)-4-(dimethylamino)but-2-enamide (pelitinib), N-(3-chlorophenyl)-6,7-dimethoxyquinazolin-4-amine (tyrphostin AG 1478), 6-(2,6-dichlorophenyl)-2-((4-(2-(diethylamino)ethoxy)phenyl)amino)-8-methylpyrido[2,3-d]pyrimidin-7(8H)-one (PD-0166285), 6-(2,6-dichlorophenyl)-8-methyl-2-((4-morpholinophenyl)amino)pyrido[2,3-d]pyrimidin-7(8H)-one (PD-173952), 6-(2,6-dichlorophenyl)-8-methyl-2-((3-(methylthio)phenyl)amino)pyrido[2,3-d]pyrimidin-7(8H)-one (PD-173955), or 6-(2,6-dichlorophenyl)-2-((4-fluoro-3-methylphenyl)amino)-8-methylpyrido[2,3-d]pyrimidin-7(8H)-one (PD-180970).

In some embodiments, the small molecule comprises a WEE1 inhibitor. In some embodiments, the WEE1 inhibitor comprises 6-(2,6-dichlorophenyl)-2-((4-(2-(diethylamino)ethoxy)phenyl)amino)-8-methylpyrido[2,3-d]pyrimidin-7(8H)-one (PD-0166285), 2-allyl-1-(6-(2-hydroxypropan-2-yl)pyridin-2-yl)-6-((4-(4-methylpiperazin-1-yl)phenyl)amino)-1,2-dihydro-3H-pyrazolo[3,4-d]pyrimidin-3-one (MK-1775), 9-hydroxy-4-phenylpyrrolo[3,4-c]carbazole-1,3(2H,6H)-dione (PD-407824), 6-butyl-4-(2-chlorophenyl)-9-hydroxypyrrolo[3,4-c]carbazole-1,3(2H,6H)-dione, or 6-(2-chloro-6-fluorophenyl)-2-((2,4,4-trimethyl-1,2,3,4-tetrahydroisoquinolin-7-yl)amino)imidazo[1,2-a]pyrimido[5,4-e]pyrimidin-5(6H)-one.

In some embodiments, the PP2A subunit is selected from the group consisting of 65 kDa regulatory subunit A alpha (PPP2R1A), 65 kDa regulatory subunit A beta (PPP2R1B), 55 kDa regulatory subunit B alpha (PPP2R2A), 55 kDa regulatory subunit B beta (PPP2R2B), 55 kDa regulatory subunit B gamma (PPP2R2C), 55 kDa regulatory subunit B delta (PPP2R2D), 72/130 kDa regulatory subunit B (PPP2R3A), 48 kDa regulatory subunit B (PPP2R3B), regulatory subunit B″ subunit gamma (PPP2R3C), regulatory subunit B′ (PPP2R4), 56 kDa regulatory subunit alpha (PPP2R5A), 56 kDa regulatory subunit beta (PPP2R5B), 56 kDa regulatory subunit gamma (PPP2R5C), 56 kDa regulatory subunit delta (PPP2R5D), 56 kDa regulatory subunit epsilon (PPP2R5E), catalytic subunit alpha (PPP2CA), and catalytic subunit beta (PPP2CB). In some embodiments, the PP2A subunit is PPP2R2A.

In some embodiments, the composition further comprises a formulation comprising at least one therapeutic agent present in an amount that is effective to cause a difference in (e.g., a decrease or increase in) expression or activity of PPP2R2A.

In some embodiments, the healthy control is from one or more subjects that do not exhibit the cancer (e.g., liver or ovarian cancer).

In some embodiments, the cancerous tissue is breast tissue. In some embodiments, the cancerous tissue is liver tissue. In some embodiments, the cancerous tissue is ovarian tissue.

In some embodiments, the formulation further comprises an excipient. In some embodiments, the excipient stabilizes the at least one therapeutic agent or provides therapeutic enhancement of the at least one therapeutic agent following administration to the subject as compared to the at least one therapeutic agent being administered to the subject in absence of the excipient.

In another aspect, disclosed herein is a kit for treating a subject having or suspected of having a cancer, comprising:

a composition comprising a formulation comprising at least one therapeutic agent, wherein the at least one therapeutic agent is present in an amount that is effective to cause a difference in expression or activity of protein kinase, membrane associated tyrosine/threonine 1 (PKMYT1) or WEE1 G2 checkpoint kinase (WEE1) following administration to the subject, and wherein the cancer is associated with cancerous tissue comprising a cell that has a difference in expression or activity level of Protein Phosphatase 2 (PP2A) or a subunit thereof as compared to a healthy control, or wherein the cancer is associated with cancerous tissue comprising a cell that displays mutations and/or deletions in genes encoding subunits of Protein Phosphatase 2 (PP2A) as compared to a healthy control; and one or more instructions for administration of the composition to the subject. In some embodiments, the difference in expression or activity level is a decrease in expression or activity level.

In some embodiments, the cancer is associated with cancerous tissue comprising a cell that has a difference in expression or activity level of Protein Phosphatase 2 (PP2A) or a subunit thereof as compared to a healthy control. In some embodiments, the difference in expression or activity level is a decrease in expression or activity level.

In some embodiments, the cancer is associated with cancerous tissue comprising a cell that displays mutations and/or deletions in genes encoding subunits of Protein Phosphatase 2 (PP2A) as compared to a healthy control. In some embodiments, the presence or absence of the mutations and/or deletions is identified by an assay of cells derived from tissue obtained from the subject. In some embodiments, the assay is a next generation sequencing-based assay.

In another aspect, disclosed herein is a method for identifying a disease in a subject, comprising assaying cells derived from tissue obtained from a subject to identify the presence or absence of mutations and/or deletions in genes encoding subunits of Protein Phosphatase 2 (PP2A) as compared to a healthy control, and outputting a report indicative of the presence or absence of mutations and/or deletions in genes encoding subunits of Protein Phosphatase 2 (PP2A) as compared to a healthy control. In some embodiments, the method comprises a next generation sequencing-based assay.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-B schematically show signaling pathways for protein kinase, membrane associated tyrosine/threonine 1 (PKMYT1, MYT1), Wee1, and Protein Phosphatase 2 55 kDa regulatory subunit B alpha (PPP2R2A).

FIG. 2 shows a plot of frequency of PPP2R2A inactivation or deficiency in different cancer types.

FIG. 3 schematically shows an example workflow for determining the effect of treatment of a population of cultured cancer cells with a nucleic acid molecule.

FIG. 4 schematically shows another example workflow for determining the effect of treatment of cultured cancer cells that are deficient in a gene using a single guide RNA that can induce a mutation in a specific gene.

FIG. 5 shows a plot of the expression level of PKMYT1 in cancer versus normal cells in various cancer types.

FIG. 6 shows a scatterplot of expression level of PKMYT1 in cancer (tumor) cells compared to normal cells.

FIGS. 7A-B show scatterplots of essentiality of PKMYT1 in two different databases (Achilles, Demeter) of cells, having inactive PPP2R2A or having wild-type PPP2R2A.

FIGS. 8A-B show example data of a CRISPR-based approach to knock out PKMYT1 and PPP2R2A in cells from two cancer types.

FIG. 9 shows HEP3B colony formation data for dual knock-out of PKMYT1 and PPP2R2A.

FIG. 10 shows the results of PKMYT1 knockout in Huh1 cells which have an endogenous deletion of the PPP2R2A gene locus.

FIG. 11 shows the results of a screen of 21 different genes to determine synthetic lethality with PKMYT1.

FIG. 12 shows the results of PKMYT1 inhibition with small molecule inhibitors in isogenic cell lines with PPP2R2A knockout.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It will be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “subject,” as used herein, generally refers to an animal, such as a mammal (e.g., human), reptile, or avian (e.g., bird), or other organism, such as a plant. For example, the subject can be a vertebrate, a mammal, a rodent (e.g., a mouse), a primate, a simian or a human. The subject can be a healthy individual, an individual that is asymptomatic with respect to a disease (e.g., liver or ovarian cancer), an individual that has or is suspected of having the disease (e.g., liver or ovarian cancer) or a pre-disposition to the disease, or an individual that is symptomatic with respect to the disease. The subject may be in need of therapy. The subject can be a patient undergoing monitoring or treatment by a healthcare provider, such as a treating physician.

The term “genome,” as used herein, generally refers to genomic information from a subject, which may be, for example, at least a portion or an entirety of a subject's hereditary information. A genome can be encoded in a deoxyribonucleic acid (DNA) molecule (s) and may be expressed in a ribonucleic acid (RNA) molecule(s). A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

Whenever a gene is referred to herein, it will be understood that a single gene can be referred to by different names. For example, “protein kinase, membrane associated tyrosine/threonine 1” and “membrane-associated tyrosine- and threonine-specific cdc2-inhibitory kinase” both refer to the same gene, PKMYT1. As another example, “protein phosphatase 2 regulatory subunit B alpha” and “serine/threonine-protein phosphatase 2A 55 kDa regulatory subunit B alpha isoform” both refer to the same gene, PPP2R2A.

### Methods for Treating Cancer

In an aspect, provided herein are methods and compositions for the treatment of cancer (e.g., liver or ovarian cancer). A method for treating a subject having or suspected of having a cancer can comprise administering to the subject a therapeutically effective amount of one or more therapeutic agents that cause a difference in (e.g., a decrease or increase in) expression or activity of one or more genes, thereby treating the subject for the cancer. The cancer may comprise a cell that has a difference in (e.g., a decrease or increase in) expression or a difference (e.g. decrease, increase or alteration) in activity level of a first gene, and administration of or exposure to the one or more therapeutic agents that cause a difference in (e.g., a decrease or increase in) expression or activity of a second gene may result in the inhibition or death of the cell. In some instances, the first gene and the second gene form a synthetic lethal gene pair. In some cases, the first gene is Protein Phosphatase 2 55 kDa regulatory subunit B alpha (PPP2R2A), and the second gene encodes a kinase, e.g., protein kinase, membrane associated tyrosine/threonine 1 (PKMYT1) or WEE1 G2 checkpoint kinase (WEE1).

In some instances, the first gene encodes for a biomarker that is deficient (e.g., under-expressed, mutated, over-expressed) in the cancer cell, and the second gene comprises a gene target to be knocked down or knocked out, thereby decreasing the expression or activity level of the second gene. In some instances, the first gene has a difference in (e.g., a decrease or increase in) expression or a difference (e.g. decrease, increase or alteration) in activity level in the cancer cell, and administration of or exposure to a therapeutically effective amount of one or more therapeutic agents that cause a difference in (e.g., a decrease or increase in) in expression or activity of the second gene in the cancer or cancer cell causes inhibition or death of the cell. In some instances, the first gene encodes a protein that regulates the cell cycle, e.g., PPP2R2A, and the second gene encodes a kinase, e.g., PKMYT1.

In some instances, the first gene display mutations and/or deletions as compared to a healthy control. The presence or absence of such mutations can be identified by assaying tissue-derived cells obtained from a subject. Appropriate assays can include those involving genomic DNA, mRNA, or cDNA. As an example, for a nucleic acid-based detection method, genomic DNA is first obtained (using any standard technique) from cells (e.g., ovarian cells) of a subject to be tested. If appropriate, cDNA can be prepared or mRNA can be obtained. In some instances, nucleic acids can be amplified by any known nucleic acid amplification technique (e.g., polymerase chain reaction) to a sufficient quantity and purity, and further analyzed to detect mutations. For example, genomic DNA can be isolated from a sample, and all exonic sequences, and the intron/exon junction regions including the regions required for exon/intron splicing, can be amplified into one or more amplicons and further analyzed for the presence or absence of mutations. In some instances, the assay is a next generation sequencing-based assay, such as FoundationOne®CDx™ or Tempus xT™.

The first gene (e.g., PPP2R2A) and the second gene (e.g., PKMYT1) may form a synthetic lethal pair, such that inhibition or decreased expression or activity level in both the first gene and the second gene is lethal to the cell (e.g., results in apoptosis, necrosis, inhibition of proliferation, etc.), but the inhibition or decreased activity of the first gene alone or the second gene alone is not sufficient to kill the cell. In some cases, inhibition or decreased expression or activity of the first gene (e.g., PPP2R2A) or the second gene (e.g., PKMYT1) alone result in a reduction in viability of a cell or cell population, but the decreased expression or activity of both genes (e.g., knockdown or knockout of PPP2R2A and PKMYT1) results in a greater reduction in viability of the cell or cell population. For example, the decrease of expression or activity of PPP2R2A and PKMYT1 may act synergistically, with a greater reduction in viability than the sum of the reductions of viability from decreased expression or activity of each member of the gene pair.

In cases where a cell (e.g., a cancerous cell) has a deficiency in the first gene (e.g., PPP2R2A), the forced decreased expression or activity level (e.g., via knock down or knock out) of the second gene (also herein “target gene,” e.g., PKMYT1) may be lethal to the cell having the deficiency in the first gene, but non-toxic or non-lethal in cells that do not have the deficiency in the first gene. Such a method of treating a subject having a cancer (e.g., liver or ovarian cancer), which cancer is associated with cancerous tissue comprising a cell having the deficiency in the first gene (e.g., PPP2R2A), using a single inhibitor (e.g., a therapeutically effective amount of a therapeutic agent that causes a decrease in expression or activity of PKMYT1) may be beneficial in reducing toxicity in normal cells of the subject and thereby reducing toxicity or side effects of cancer treatment.

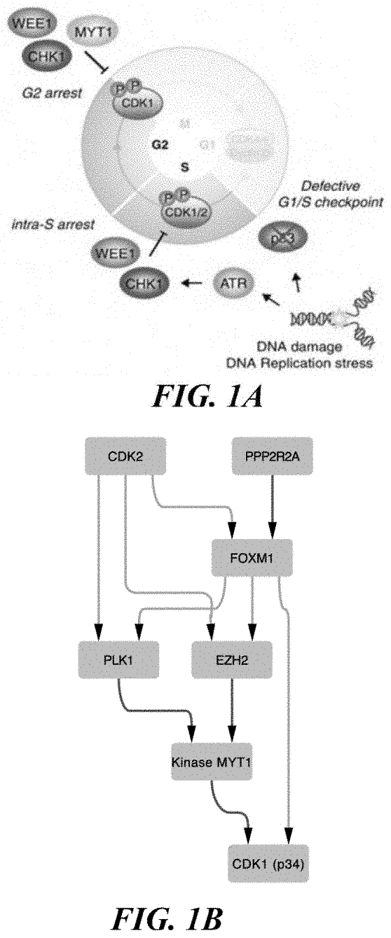

In some cases, the first gene may be or encode a protein that is an upstream agonist or antagonist of the second gene, or the second gene may be or encode a protein that is an upstream agonist or antagonist of the first gene. By way of example, the first gene may be PPP2R2A and the second gene may be PKMYT1. PPP2R2A, when expressed in a normal (e.g., non-mutated) cell, can act as an indirect positive regulator of PKMYT1, which is a kinase that is an upstream regulator of various proteins within a protein signaling cascade or signal transduction pathway (see, FIGS. 1A-B). For instance, PKMYT1 can interact with or regulate CDK1 and thereby affect cell cycle progression. In normal or non-cancerous cells, expression of PPP2R2A can positively regulate PKMYT1, thus indirectly inhibiting CDK1 and preventing uncontrolled cell cycle progression. PPP2R2A expression is also known to promote DNA repair (Cancer Res. 2012 Dec. 15; 72(24):6414-24.). Hence, in cells where PPP2R2A is deficient (e.g., a cell that has a mutated PPP2R2A), damaged DNA may go unrepaired, and PKMYT1 may not be inhibited, thereby causing increased expression of the downstream protein CDK1. Accordingly, PKMYT1 inhibition in PPP2R2A-deficient cells can lead to uncontrolled cell cycle progression for cells with damaged DNA and thus induce cell death.

Although PPP2R2A and PKMYT1 are shown as examples, other gene interactions can be possible. In one such example, the first gene may be an agonist or antagonist of another gene (or encoded protein) that regulates the second gene, or the second gene may be an agonist or antagonist of another gene or encoded protein that regulates the first gene. Similarly, the first gene may be an agonist or antagonist of another gene (or encoded protein) that regulates yet another gene (or encoded protein) that may regulate the second gene, or the second gene may be an agonist or antagonist of another gene (or encoded protein) that regulates yet another gene (or encoded protein) that may regulate the first gene. In some cases, the first or second gene may regulate another gene or protein that is at least 1, 2, 3, 4, 5, 6, 7, 8, or more components (e.g., nodes or other genes, proteins, or signal transducers) upstream of the second or first gene, respectively.

In some instances, the first gene and the second gene may regulate a subset of the same genes downstream. For example, the first gene may regulate a plurality of downstream genes, a subset of which are also regulated by the second gene. In cancer cells, the downstream genes may comprise genes important in cancer-related processes, e.g., HIPPO pathway, epithelial-to-mesenchymal transition, P13K pathway, DNA replication, cell migration, cell metastasis, etc. Alternatively or in addition to, the first gene and the second gene may be regulated by a subset of the same genes.

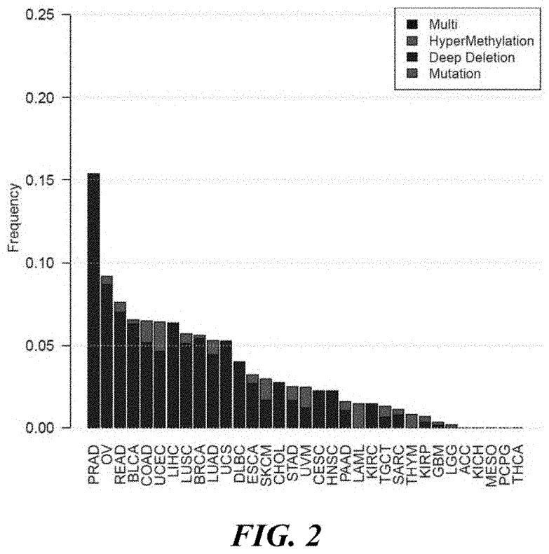

In some cases, the first gene or the second gene may also be a biomarker for a cancer (e.g., liver or ovarian cancer). For instance, the first gene may be PPP2R2A. In some cases, in a cancer cell, PPP2R2A may be lowly expressed, mutated, or otherwise deficient in a cancer cell when compared to a control cell or population of cells. For instance, FIG. 2 shows a plot of frequency (Y-axis) of PPP2R2A inactivation or deficiency in different cancer types (X-axis). In certain types of cancers (e.g., prostate adenocarcinoma), the frequency of mutation of PPP2R2A can be as high as about 15%. In certain other cancer types (e.g., ovarian serous cystadenocarcinoma, rectum adenocarcinoma), the frequency of mutation of PPP2R2A may be greater than 10%. In various cancer types, the deficiency of PPP2R2A leading to inactivation may include: multiple copies of the same gene, hypermethylation, deep deletion, or mutation in the PPP2R2A gene. In cancers comprising the PPP2R2A mutation, administration of or exposure to a therapeutically effective amount of one or more therapeutic agents that causes the decrease in expression or activity of PKMYT1 may result in synthetic lethality of the PPP2R2A-mutated cells.

In some cases, the one or more therapeutic agents used to cause a difference in (e.g., a decrease or increase in) in expression or activity of the second gene (e.g., PKMYT1) may comprise a small molecule (e.g., a molecule having a molecular weight of less than 900 Daltons), a protein, an intrabody, a peptide, a ribonucleic acid (RNA) molecule, a deoxyribonucleic acid (DNA) construct, or a combination thereof (e.g., a protein-nucleic acid complex). In an example, the one or more therapeutic agents may comprise a protein-nucleic acid complex, e.g., an endonuclease complex and a DNA construct. In some cases, the endonuclease complex comprises a clustered regularly interspaced short palindromic repeat (CRISPR) associated (Cas) protein or variant thereof (e.g., an engineered variant). In such cases, the DNA construct may be co-administered with the endonuclease complex. Alternatively or in addition to, the DNA construct may comprise an endonuclease gene. In such instances, the DNA construct may comprise a gene encoding for a Cas protein or variant thereof (e.g., an engineered variant). After the DNA construct is introduced or delivered to a cell (e.g., cancer cell), the DNA construct may be transcribed and translated by the cell using the cell's own machinery (e.g., polymerases, ribosomes, etc.).

In some instances, the one or more therapeutic agents used to cause a difference in (e.g., a decrease or increase in) in expression or activity of a target gene (e.g., PKMYT1) comprises a small molecule inhibitor (e.g., a molecule having a molecular weight of less than 900 Daltons). The small molecule may be configured to decrease the expression level or activity level of the target gene alone, or the small molecule may be configured to decrease the expression level or activity level of the target gene in combination with the deficient or mutated gene (e.g., PPP2R2A in a cancer cell). In some cases, the small molecule may directly interact with both the first gene and the second gene. For example, the small molecule may inhibit the protein or proteins encoded by one or both of the first gene and the second gene, respectively. Alternatively or in addition to, the small molecule may inhibit an upstream effector or downstream protein in a signaling pathway in which one or both of the genes interact.

In some cases, the small molecule inhibitor may comprise an PKMYT1 inhibitor. The PKMYT1 inhibitor may be, for example, 5-((5-methoxy-2-((4-morpholinophenyl)amino)pyrimidin-4-yl)amino)-2-methylphenol, N-(2-chloro-6-methylphenyl)-2-((6-(4-(2-hydroxyethyl)piperazin-1-yl)-2-methylpyrimidin-4-yl)amino)thiazole-5-carboxamide (dasatinib), 4-((2,4-dichloro-5-methoxyphenyl)amino)-6-methoxy-7-(3-(4-methylpiperazin-1-yl)propoxy)quinoline-3-carbonitrile (bosutinib), N-(5-chlorobenzo[d][1,3]dioxol-4-yl)-7-(2-(4-methylpiperazin-1-yl)ethoxy)-5-((tetrahydro-2H-pyran-4-yl)oxy)quinazolin-4-amine (saracatinib), (E)-N-(4-((3-chloro-4-fluorophenyl)amino)-3-cyano-7-ethoxyquinolin-6-yl)-4-(dimethylamino)but-2-enamide (pelitinib), N-(3-chlorophenyl)-6,7-dimethoxyquinazolin-4-amine (tyrphostin AG 1478), 6-(2,6-dichlorophenyl)-2-((4-(2-(diethylamino)ethoxy)phenyl)amino)-8-methylpyrido[2,3-d]pyrimidin-7(8H)-one (PD-0166285), 6-(2,6-dichlorophenyl)-8-methyl-2-((4-morpholinophenyl)amino)pyrido[2,3-d]pyrimidin-7(8H)-one (PD-173952), 6-(2,6-dichlorophenyl)-8-methyl-2-((3-(methylthio)phenyl)amino)pyrido[2,3-d]pyrimidin-7(8H)-one (PD-173955), or 6-(2,6-dichlorophenyl)-2-((4-fluoro-3-methylphenyl)amino)-8-methylpyrido[2,3-d]pyrimidin-7(8H)-one (PD-180970). The small molecule inhibitor may be configured to inhibit or decrease the expression of PKMYT1 (the gene) or the activity of protein kinase, membrane associated tyrosine/threonine 1 (a protein derived from the PKMYT1 gene), either directly or indirectly. For instance, the small molecule inhibitor may inhibit the protein kinase, membrane associated tyrosine/threonine 1 protein or another protein that may be upstream or downstream of protein kinase, membrane associated tyrosine/threonine 1 in a signaling pathway, such as, but not limited to, those shown in FIGS. 1A-B. For example, the small molecule inhibitor may inhibit or otherwise decrease the expression or activity level of WEE1, CHK1, CDK1, CDK2, PPP2R2A, FOXM1, PLK1, EZH2, etc.

In some cases, the small molecule inhibitor may comprise a WEE1 inhibitor. The WEE1 inhibitor may be, for example, 6-(2,6-dichlorophenyl)-2-((4-(2-(diethylamino)ethoxy)phenyl)amino)-8-methylpyrido[2,3-d]pyrimidin-7(8H)-one (PD-0166285), 2-allyl-1-(6-(2-hydroxypropan-2-yl)pyridin-2-yl)-6-((4-(4-methylpiperazin-1-yl)phenyl)amino)-1,2-dihydro-3H-pyrazolo[3,4-d]pyrimidin-3-one (MK-1775), 9-hydroxy-4-phenylpyrrolo[3,4-c]carbazole-1,3(2H,6H)-dione (PD-407824), 6-butyl-4-(2-chlorophenyl)-9-hydroxypyrrolo[3,4-c]carbazole-1,3(2H,6H)-dione, or 6-(2-chloro-6-fluorophenyl)-2-((2,4,4-trimethyl-1,2,3,4-tetrahydroisoquinolin-7-yl)amino)imidazo[1,2-a]pyrimido[5,4-e]pyrimidin-5(6H)-one. The small molecule inhibitor may be configured to inhibit or decrease the expression of WEE1 (the gene) or the activity of WEE1 G2 checkpoint kinase (a protein derived from the WEE1 gene), either directly or indirectly. For instance, the small molecule inhibitor may inhibit the WEE1 G2 checkpoint kinase protein or another protein that may be upstream or downstream of WEE1 G2 checkpoint kinase in a signaling pathway, such as, but not limited to, those shown in FIG. 1A. For example, the small molecule inhibitor may inhibit or otherwise decrease the expression or activity level of CDK1, CDK2, etc.

In some cases, the small molecule inhibitor may comprise a combination of small molecule inhibitors or derivatives thereof. For example, a small molecule inhibitor may be engineered or modified for dual specificity and may decrease expression or activity of both the first gene and the second gene (e.g., PKMYT1 and PPP2R2A). Alternatively or in addition to, a combination of small molecule inhibitors (e.g., a small molecule “cocktail”) may be used to decrease expression or activity of the target gene (e.g., PKMYT1) alone or both the first gene and the second gene. In some cases, a small molecule inhibitor may be administered with another agent type (e.g., protein, RNA molecule, DNA molecule, etc.).

The small molecule inhibitor may be administered in any useful concentration. For example, a small molecule may be administered at a concentration of about 0.5 nanomolar (nM), about 1 nM, about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 micromolar (04), about 2 μM, about 3 μM, about 4 μM, about 5 μM, about 6 μM, about 7 μM, about 8 μM, about 9 μM, about 10 μM. A small molecule may be administered at a concentration of at least about 0.5 nanomolar (nM), at least about 1 nM, at least about 10 nM, at least about 20 nM, at least about 30 nM, at least about 40 nM, at least about 50 nM, at least about 60 nM, at least about 70 nM, at least about 80 nM, at least about 90 nM, at least about 100 nM, at least about 200 nM, at least about 300 nM, at least about 400 nM, at least about 500 nM, at least about 600 nM, at least about 700 nM, at least about 800 nM, at least about 900 nM, at least about 1 micromolar (04), at least about 2 μM, at least about 3 μM, at least about 4 μM, at least about 5 μM, at least about 6 μM, at least about 7 μM, at least about 8 μM, at least about 9 μM, at least about 10 μM. A small molecule may be administered at a concentration of at most about 10 μM, at most about 9 μM, at most about 8 μM, at most about 7 μM, at most about 6 μM, at most about 5 μM, at most about 4 μM, at most about 3 μM, at most about 2 μM, at most about 1 μM, at most about 900 nM, at most about 800 nM, at most about 700 nM, at most about 600 nM, at most about 500 nM, at most about 400 nM, at most about 300 nM, at most about 200 nM, at most about 100 nM, at most about 90 nM, at most about 80 nM, at most about 70 nM, at most about 60 nM, at most about 50 nM, at most about 40 nM, at most about 30 nM, at most about 20 nM, at most about 10 nM, at most about 1 nM, at most about 0.5 nM, etc. A range of concentrations may be used, e.g., between 22 nM-1 μM. Where more than one small molecule is used, the concentrations may be the same of different for each small molecule used.

In some cases, the small molecule inhibitor may be configured to have higher selectivity for PKMYT1 over a similar gene (e.g., WEE1, etc.). The small molecule inhibitor may have a higher selectivity for PKMYT1 over a similar gene by about 1 fold, 2 fold, 3 fold, 4 fold, 5 fold, 6 fold, 7 fold, 8 fold, 9 fold, 10 fold, 20 fold, 30 fold, 40 fold, 50 fold, 60 fold, 70 fold, 80 fold, 90 fold, 100 fold, 200 fold, 300 fold, 400 fold, 500 fold, or more. The small molecule inhibitor may have a higher selectivity for PKMYT1 over a similar gene by at least 1 fold, at least 2 fold, at least 3 fold, at least 4 fold, at least 5 fold, at least 6 fold, at least 7 fold, at least 8 fold, at least 9 fold, at least 10 fold, at least 20 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, or more.

In cancers comprising a deficiency in the first gene (e.g., PPP2R2A), one or more therapeutic agents used to cause a difference in (e.g., a decrease or increase in) expression or activity of the target gene (e.g., PKMYT1) may require a lower concentration or dosage to be delivered to a subject for therapeutic efficacy. For instance, PPP2R2A and PKMYT1 may be synthetic lethal, and administration of an PKMYT1 inhibitor to a subject having a cancer cell that has a deficiency in PPP2R2A may be therapeutically effective. In such an example, a lower dosage of PKMYT1 inhibitor may be sufficient to kill the PPP2R2A-deficient cancer cells, compared to cells (e.g., non-cancer cells) that do not have the PPP2R2A deficiency. As higher dosages or concentrations of PKMYT1 inhibition in a subject may increase toxicity, administration of a lower concentration or dosage of PKMYT1 inhibitor in selected or pre-screened cancer types (e.g., cancers comprising the PPP2R2A mutation) may be advantageous to reduce toxicity and side effects to the subject.

In some cases, the method for treating the subject having a cancer (e.g., liver or ovarian cancer) further comprises administering to the subject a therapeutically effective amount of one or more therapeutic agents that causes a difference in (e.g., a decrease or increase in) expression or activity of PPP2R2A. In some cases, the method for treating the subject having a cancer further comprises administering to the subject a therapeutically effective amount of one or more therapeutic agents that causes a decrease in expression or activity of CDK1.

In some cases, the one or more therapeutic agent used to cause a difference in (e.g., a decrease or increase in) expression or activity of the target gene comprises a DNA construct. By way of example, the target gene may be PKMYT1. The DNA construct may comprise a guide RNA (gRNA) sequence, which may be used to direct a protein (e.g., Cas protein) to the target gene (e.g., PKMYT1). The DNA construct may comprise a gRNA sequence, which may direct the protein (e.g., Cas protein) to a target gene (e.g., PKMYT1). The DNA construct may comprise an RNA sequence, a DNA sequence, or a combination thereof. In some cases, the DNA construct comprises: (i) a first gRNA sequence, which may be used to direct an endonuclease (e.g., Cas protein) to a targeted location or gene locus for a target gene (e.g., PKMYT1) and (ii) a first sequence (e.g., a DNA sequence) corresponding to the gene (e.g., a gene replacement for PKMYT1). It will be appreciated that different combinations of RNA sequences and DNA sequences may be used in the DNA construct. Moreover, other functional sequences may be included in the DNA sequence, including, but not limited to, a barcode sequence, a tag, or other identifying sequence, a primer sequence, a restriction site, a transposition site, etc.

The endonuclease complex may comprise an endonuclease, e.g., a Cas protein, or other nucleic acid-interacting enzyme (e.g., ligase, helicase, reverse transcriptase, transcriptase, polymerase, etc.). The Cas protein may comprise any Cas type (e.g., Cas I, Cas IA, Cas IB, Cas IC, Cas ID, Cas IE, Cas IF, Cas IU, Cas III, Cas IIIA, Cas IIIB, Cas IIIC, Cas IIID, Cas IV, Cas IVA, Cas IVB, Cas II, Cas IIA, Cas IIB, Cas IIC, Cas V, Cas VI). In some instances, the Cas protein may comprise other proteins (e.g., a fusion protein) and may comprise an additional enzyme that may associate with a nucleic acid molecule (e.g., ligase, transcriptase, transposase, nuclease, endonuclease, reverse transcriptase, polymerase, helicase, etc.). The endonuclease complex may be delivered exogenously or may be encoded in the DNA construct for transcription and translation within the cell.

In some cases, the one or more therapeutic agents used to cause a difference in (e.g., a decrease or increase in) expression in the target gene (e.g., PKMYT1) may comprise a protein or peptide. For example, the one or more therapeutic agents may comprise an antibody, an antibody fragment, a hormone, a ligand, or an immunoglobulin. The protein or peptide may be naturally occurring or may be synthetic. The protein may be an engineered variant of a protein (e.g., recombinant protein), or fragment thereof. The protein may be subjected to other modifications, e.g., post-translational modifications, including but not limited to: glycosylation, acylation, prenylation, lipoylation, alkylation, amidation, acetylation, methylation, formylation, butyrylation, carboxylation, phosphorylation, malonylation, hydroxylation, iodination, propionylation, S-nitrosylation, S-glutationylation, succinylation, sulfation, glycation, carbamylation, carbonylation, biotinylation, carbamylation, oxidation, pegylation, sumoylation, ubiquitination, ubiquitylation, racemization, etc. One or more modifications may be made to the protein or peptide.

In some cases, the one or more therapeutic agents used to cause a difference in (e.g., a decrease or increase in) expression or activity of the target gene (e.g., PKMYT1) may comprise a nucleic acid molecule, e.g., an RNA molecule. The RNA molecule can comprise any suitable RNA molecule and size sufficient to decrease the expression level or activity of the target gene (e.g., PKMYT1). The RNA molecule may comprise a small hairpin RNA (shRNA) molecule, a small interfering RNA (siRNA), a microRNA (miRNA), or other useful RNA molecule. In some examples, the RNA molecule may comprise a messenger RNA (mRNA), transfer RNA (tRNA), ribosomal RNAs (rRNA), small nuclear RNA (snRNA), piwi-interacting (piRNA), non-coding RNA (ncRNA), long non-coding RNA, (lncRNA), and fragments of any of the foregoing. The RNA molecule may be single-stranded, double-stranded, or partially single- or double-stranded.

It will be appreciated that one or more therapeutic agents (e.g., peptides, RNA molecules, protein-nucleic acid complexes) are listed as examples and that a combination of therapeutic agent types may be used to treat the subject. For instance, administering one or more different types of therapeutic agents may be used to decrease the expression or activity of the target gene (e.g., PKMYT1). For example, a protein or peptide may be co-administered with a small molecule (e.g., a molecule having a molecular weight of less than 900 Daltons), an RNA molecule, a DNA molecule, or a complexed molecule (e.g., protein-nucleic acid molecule). Similarly, an RNA molecule may be administered with a small molecule, a DNA molecule, or a complexed molecule. In another example, a small molecule may be co-administered with a DNA molecule or a complexed molecule. Any of these combinations may be used to decrease the expression or activity of the target gene (PKMYT1) in a cell comprising a mutation in the first gene (e.g., PPP2R2A). These combinations are non-limiting examples of different combinations of agents that may be used to treat the subject having or suspected of having cancer (e.g., liver or ovarian cancer).

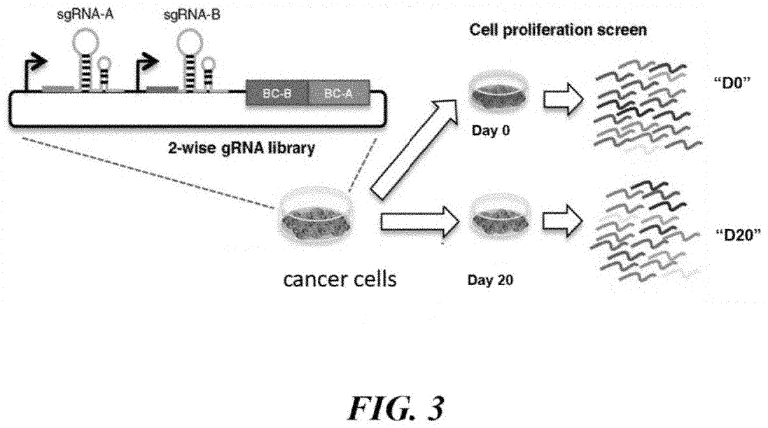

For example, FIG. 3 schematically illustrates an example workflow for determining the effect of treatment of a population of cultured cancer cells with a pair of guide RNAs targeting two different genes. In such an example, the treatment may comprise administration of a nucleic acid molecule to decrease the activity or expression of the first gene (e.g., PPP2R2A) and the second gene (e.g., PKMYT1). The nucleic acid molecule can comprise a DNA construct, which may comprise a first gRNA sequence (sgRNA-A), a second gRNA sequence (sgRNA-B), a first DNA sequence (BC-B) and a second DNA sequence (BC-A). The first DNA sequence or the second DNA sequence, or both the first and the second DNA sequences may comprise a barcode sequence. The first guide sequence may have sequence homology to the first gene (e.g., PPP2R2A) and thus may target the first gene for mutagenesis by a protein (e.g., an endonuclease, e.g., Cas9), and the second guide sequence may have sequence homology to the second gene (e.g., PKMYT1) and thus may target the second gene for mutagenesis by a protein (e.g., an endonuclease, e.g., Cas9). Cells (e.g., cancer cells) may be treated with a therapeutically effective amount of the DNA construct and a protein (e.g., Cas9). In some cases, the DNA construct may be introduced via transfection (e.g., using a liposome or other nanoparticle) or transduction (e.g., using a virus). The protein may be administered using a nanoparticle or other vesicle, or by adding the protein to the cell culture media. The protein (e.g., Cas9) may use the sgRNA-A and sgRNA-B to direct the protein to a specific locus or location in the cell genome (e.g., at a locus of PPP2R2A and PKMYT1). Next, the protein may excise and/or replace the endogenous genes (e.g., PPP2R2A and PKMYT1). If replacing the endogenous genes, the protein (e.g., Cas9) may replace the endogenous genes with the first DNA sequence (BC-B) and the second DNA sequence (BC-A). Cells may then be cultivated for a duration of time (e.g., 7 days, 14 days, 20 days, etc.). The DNA from the population of cells that has been cultivated can be sequenced to establish the abundance of each of the possible pairs of guide RNA present. A substantial reduction in the abundance of a specific pair of guides may suggest that that combination of gene knock-downs has a deleterious effect on the ability of those cells to proliferate.

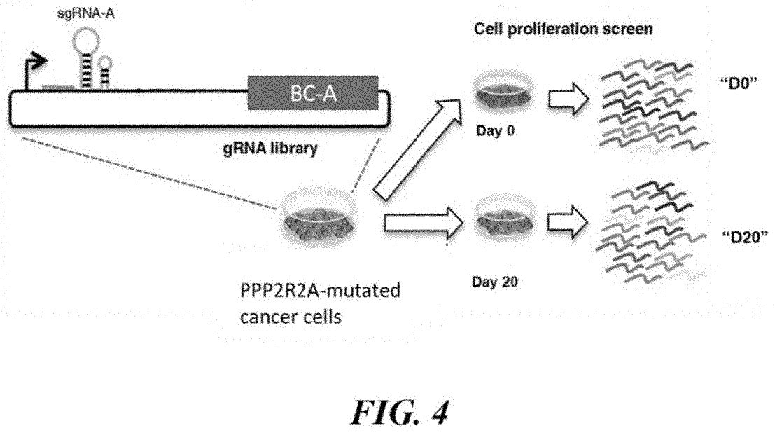

In some cases, only the target gene may be knocked out in a cell or population of cells, which cell comprises a deficient gene that is synthetic lethal with the target gene. FIG. 4 schematically illustrates an example workflow for determining the effect of treatment of a population of cultured cancer cells that are deficient in a gene (e.g., PPP2R2A). In such an example, the treatment may comprise administration of a nucleic acid molecule to decrease the activity or expression of the target gene (e.g., PKMYT1). The nucleic acid molecule can comprise a DNA construct, which may comprise a gRNA sequence (sgRNA-A) and a DNA sequence (BC-A). The DNA sequence may comprise a barcode sequence, and the guide sequence may have sequence homology to the target gene (e.g., PKMYT1) and thus may target the target gene for mutagenesis by a protein (e.g., an endonuclease, e.g., Cas9). A population of cells (e.g., cancer cells) comprising the mutation (e.g., PPP2R2A mutation) may be treated with a therapeutically effective amount of the DNA construct and a protein (e.g., Cas9). In some cases, the DNA construct may be introduced via transfection (e.g., using a liposome or other nanoparticle) or transduction (e.g., using a virus). The protein may be administered using a nanoparticle or other vesicle, or by adding the protein to the cell culture media. The protein (e.g., Cas9) may use the sgRNA-A to direct the protein to a specific locus or location in the cell genome (e.g., at a locus of PKMYT1). Next, the protein may excise and/or replace the endogenous genes (e.g., PKMYT1). If replacing the endogenous genes, the protein (e.g., Cas9) may replace the endogenous genes with the DNA sequence (BC-A). Cells may then be cultivated for a duration of time (e.g., 7 days, 14 days, 20 days, etc.). The proliferation or viability of the cells may be measured, and in some instances, compared to a control population of cells (e.g., non-mutant PPP2R2A cells). The DNA from the population of cells that has been cultivated can be sequenced to establish the abundance of each of the possible guide RNAs present. A substantial reduction in the abundance of a specific guide may suggest that that gene knock-down has a deleterious effect on the ability of those cells to proliferate. Guide RNAs that reduce the proliferation of PPP2R2A mutant cells, but not wild type cells, may be considered to have synthetic lethality with PPP2R2A.

In some cases, the deficient gene that is synthetic lethal with the target gene is the gene encoding one of the subunits of PP2A. In some cases, the PP2A subunit is selected from the group consisting of 65 kDa regulatory subunit A alpha (PPP2R1A), 65 kDa regulatory subunit A beta (PPP2R1B), 55 kDa regulatory subunit B alpha (PPP2R2A), 55 kDa regulatory subunit B beta (PPP2R2B), 55 kDa regulatory subunit B gamma (PPP2R2C), 55 kDa regulatory subunit B delta (PPP2R2D), 72/130 kDa regulatory subunit B (PPP2R3A), 48 kDa regulatory subunit B (PPP2R3B), regulatory subunit B″ subunit gamma (PPP2R3C), regulatory subunit B′ (PPP2R4), 56 kDa regulatory subunit alpha (PPP2R5A), 56 kDa regulatory subunit beta (PPP2R5B), 56 kDa regulatory subunit gamma (PPP2R5C), 56 kDa regulatory subunit delta (PPP2R5D), 56 kDa regulatory subunit epsilon (PPP2R5E), catalytic subunit alpha (PPP2CA), and catalytic subunit beta (PPP2CB). In some cases, the subunit is PPP2R2A.

In another aspect, disclosed herein is a composition for treating a cancer (e.g., liver or ovarian cancer), comprising a formulation comprising (i) at least one therapeutic agent and (ii) an excipient, wherein the at least one therapeutic agent is present in an amount that is effective to cause a difference in (e.g., a decrease or increase in) expression or activity of protein kinase, membrane associated tyrosine/threonine 1 (PKMYT1) following administration or exposure to the subject, wherein the excipient stabilizes the at least one therapeutic agent or provides therapeutic enhancement of the at least one therapeutic agent following administration or exposure to the subject as compared to the at least one therapeutic agent being administered to the subject in absence of the excipient, and wherein the cancer is associated with cancerous tissue comprising a cell that has a difference in (e.g., a decrease or increase in) expression or a difference (e.g. decrease, increase or alteration) in activity level of Protein Phosphatase 2 (PP2A) or a subunit thereof as compared to a healthy control.

In some cases, the cancerous tissue is breast tissue, pancreatic tissue, uterine tissue, bladder tissue, colorectal tissue, prostate tissue, liver tissue, or ovarian tissue. In some cases, the cancerous tissue is liver tissue. In some case, the cancerous tissue is ovarian tissue.

In some cases, the at least one therapeutic agent used to cause a difference in (e.g., a decrease or increase in) expression or activity of PKMYT1 may comprise a small molecule (e.g., a molecule having a molecular weight of less than 900 Daltons), a protein, a peptide, a ribonucleic acid (RNA) molecule, a deoxyribonucleic acid (DNA) construct, or a combination thereof (e.g., a protein-nucleic acid complex). In an example, the at least one therapeutic agent may comprise a protein-nucleic acid complex, e.g., an endonuclease complex and a DNA construct. In some cases, the endonuclease complex comprises a clustered regularly interspaced short palindromic repeat (CRISPR) associated (Cas) protein or variant thereof (e.g., an engineered variant). In such cases, the DNA construct may be co-administered with the endonuclease complex. Alternatively or in addition to, the DNA construct may comprise an endonuclease gene. In such instances, the DNA construct may comprise a gene encoding for a Cas protein or variant thereof (e.g., an engineered variant). After the DNA construct is introduced or delivered to a cell (e.g., cancer cell), the DNA construct may be transcribed and translated by the cell using the cell's own machinery (e.g., polymerases, ribosomes, etc.).

In some instances, the at least one therapeutic agent used to cause a difference in (e.g., a decrease or increase in) expression or activity of PKMYT1 comprises a small molecule inhibitor (e.g., a molecule having a molecular weight of less than 900 Daltons). The small molecule may be configured to decrease the expression level or activity level of the target gene alone, or the small molecule may be configured to decrease the expression level or activity level of the PKMYT1 and PPP2R2A. In some cases, the small molecule may directly interact with PKMYT1, or PKMYT1 and PPP2R2A. For example, the small molecule may inhibit the protein or proteins encoded by PKMYT1 alone, or the combination of PKMYT1 and PPP2R2A, respectively. Alternatively or in addition to, the small molecule may inhibit an upstream effector or downstream protein in a signaling pathway in which PKMYT1 or PPP2R2A interact.

In some cases, the small molecule inhibitor may comprise an PKMYT1 inhibitor. The PKMYT1 inhibitor may be, for example, dasatinib, saracatinib, pelitinib, tyrphostin AG 1478, PD-0166285, PD-173952, PD-173955, or PD-180970. The small molecule inhibitor may be configured to inhibit or decrease the expression of PKMYT1 (the gene) or the activity of protein kinase, membrane associated tyrosine/threonine 1 (a protein derived from the PKMYT1 gene) directly or indirectly. For instance, the small molecule inhibitor may inhibit the protein kinase, membrane associated tyrosine/threonine 1 protein or another protein that may be upstream or downstream of protein kinase, membrane associated tyrosine/threonine 1 in a signaling pathway, such as, but not limited to, those shown in FIGS. 1A-B.

In some cases, the small molecule inhibitor may comprise a WEE1 inhibitor. The WEE1 inhibitor may be, for example, 6-(2,6-dichlorophenyl)-2-((4-(2-(diethylamino)ethoxy)phenyl)amino)-8-methylpyrido[2,3-d]pyrimidin-7(8H)-one (PD-0166285), 2-allyl-1-(6-(2-hydroxypropan-2-yl)pyridin-2-yl)-6-((4-(4-methylpiperazin-1-yl)phenyl)amino)-1,2-dihydro-3H-pyrazolo[3,4-d]pyrimidin-3-one (MK-1775), 9-hydroxy-4-phenylpyrrolo[3,4-c]carbazole-1,3(2H,6H)-dione (PD-407824), 6-butyl-4-(2-chlorophenyl)-9-hydroxypyrrolo[3,4-c]carbazole-1,3(2H,6H)-dione, or 6-(2-chloro-6-fluorophenyl)-2-((2,4,4-trimethyl-1,2,3,4-tetrahydroisoquinolin-7-yl)amino)imidazo[1,2-a]pyrimido[5,4-e]pyrimidin-5(6H)-one. The small molecule inhibitor may be configured to inhibit or decrease the expression of WEE1 (the gene) or the activity of WEE1 G2 checkpoint kinase (a protein derived from the WEE1 gene), either directly or indirectly. For instance, the small molecule inhibitor may inhibit the WEE1 G2 checkpoint kinase protein or another protein that may be upstream or downstream of WEE1 G2 checkpoint kinase in a signaling pathway, such as, but not limited to, those shown in FIG. 1A. For example, the small molecule inhibitor may inhibit or otherwise decrease the expression or activity level of CDK1, CDK2, etc.

In some cases, the small molecule inhibitor may comprise a combination of small molecule inhibitors or derivatives thereof. For example, a small molecule inhibitor may be engineered or modified for dual specificity and may decrease expression or activity of both PKMYT1 and PPP2R2A. Alternatively or in addition to, a combination of small molecule inhibitors (e.g., a small molecule “cocktail”) may be used to decrease expression of PKMYT1, or the combination of PKMYT1 and PPP2R2A. In some cases, a small molecule inhibitor may be administered with another therapeutic agent type (e.g., protein, RNA molecule, DNA molecule, etc.).

The small molecule inhibitor may be administered in any useful concentration. For example, a small molecule may be administered at a concentration of about 0.5 nanomolar (nM), about 1 nM, about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 micromolar (μM), about 2 μM, about 3 μM, about 4 μM, about 5 μM, about 6 μM, about 7 μM, about 8 μM, about 9 μM, about 10 μM. A small molecule may be administered at a concentration of at least about 0.5 nanomolar (nM), at least about 1 nM, at least about 10 nM, at least about 20 nM, at least about 30 nM, at least about 40 nM, at least about 50 nM, at least about 60 nM, at least about 70 nM, at least about 80 nM, at least about 90 nM, at least about 100 nM, at least about 200 nM, at least about 300 nM, at least about 400 nM, at least about 500 nM, at least about 600 nM, at least about 700 nM, at least about 800 nM, at least about 900 nM, at least about 1 micromolar (μM), at least about 2 μM, at least about 3 μM, at least about 4 μM, at least about 5 μM, at least about 6 μM, at least about 7 μM, at least about 8 μM, at least about 9 μM, at least about 10 μM. A small molecule may be administered at a concentration of at most about 10 μM, at most about 9 μM, at most about 8 μM, at most about 7 μM, at most about 6 μM, at most about 5 μM, at most about 4 μM, at most about 3 μM, at most about 2 μM, at most about 1 μM, at most about 900 nM, at most about 800 nM, at most about 700 nM, at most about 600 nM, at most about 500 nM, at most about 400 nM, at most about 300 nM, at most about 200 nM, at most about 100 nM, at most about 90 nM, at most about 80 nM, at most about 70 nM, at most about 60 nM, at most about 50 nM, at most about 40 nM, at most about 30 nM, at most about 20 nM, at most about 10 nM, at most about 1 nM, at most about 0.5 nM, etc. A range of concentrations may be used, e.g., between 22 nM-1 μM. Where more than one small molecule is used, the concentrations may be the same of different for each small molecule used. As described elsewhere herein, a lower concentration or dosage of the one or more therapeutic agents to inhibit PKMYT1 may be therapeutically effective in cancers that comprise a cell having a PPP2R2A deficiency, as compared to non-deficient cancer cells.

In some cases, the composition may further comprise at least one therapeutic agent present in an amount that is effective in causing a difference in (e.g., a decrease or increase in) expression or activity of PPP2R2A. In some cases, the method for treating the subject having a cancer (e.g., liver or ovarian cancer) further comprises administering to the subject a therapeutically effective amount of one or more therapeutic agents that causes a difference in (e.g., a decrease or increase in) expression or activity of CDK1.

In some cases, the one or more therapeutic agent used to cause a difference in (e.g., a decrease or increase in) expression PKMYT1 comprises a DNA construct. The DNA construct may comprise a guide RNA (gRNA) sequence, which may be used to direct a protein (e.g., Cas protein) to the PKMYT1. The DNA construct may comprise a gRNA sequence, which may direct the protein (e.g., Cas protein) to PKMYT1. The DNA construct may comprise an RNA sequence, a DNA sequence, or a combination thereof. In some cases, the DNA construct comprises: (i) a first gRNA sequence, which may be used to direct an endonuclease (e.g., Cas protein) to a targeted location or gene locus for PKMYT1 and (ii) a sequence (e.g., a DNA sequence) corresponding to the PKMYT1 gene (e.g., a gene replacement for PKMYT1). It will be appreciated, that different combinations of RNA sequences and DNA sequences may be used in the DNA construct. Moreover, other functional sequences may be included in the DNA sequence, including, but not limited to, a barcode sequence, a tag, or other identifying sequence, a primer sequence, a restriction site, a transposition site, etc.

The endonuclease complex may comprise an endonuclease, e.g., a Cas protein, or other nucleic acid-interacting enzyme (e.g., ligase, helicase, reverse transcriptase, transcriptase, polymerase, etc.). The Cas protein may comprise any Cas type (e.g., Cas I, Cas IA, Cas IB, Cas IC, Cas ID, Cas IE, Cas IF, Cas IU, Cas III, Cas IIIA, Cas IIIB, Cas IIIC, Cas IIID, Cas IV, Cas IVA, Cas IVB, Cas II, Cas IIA, Cas JIB, Cas IIC, Cas V, Cas VI). In some instances, the Cas protein may comprise other proteins (e.g., a fusion protein) and may comprise an additional enzyme that may associate with a nucleic acid molecule (e.g., ligase, transcriptase, transposase, nuclease, endonuclease, reverse transcriptase, polymerase, helicase, etc.). The endonuclease complex may be delivered exogenously or may be encoded in the DNA construct for transcription and translation within the cell.

In some cases, the at least one therapeutic agent used to cause a difference in (e.g., a decrease or increase in) expression in PKMYT1 may comprise a protein or peptide. For example, the one or more therapeutic agent may comprise an antibody, an antibody fragment, a hormone, a ligand, or an immunoglobulin. The protein or peptide may be naturally occurring or may be synthetic. The protein may be an engineered variant of a protein (e.g., recombinant protein), or fragment thereof. The protein may be subjected to other modifications, e.g., post-translational modifications, including but not limited to: glycosylation, acylation, prenylation, lipoylation, alkylation, amidation, acetylation, methylation, formylation, butyrylation, carboxylation, phosphorylation, malonylation, hydroxylation, iodination, propionylation, S-nitrosylation, S-glutationylation, succinylation, sulfation, glycation, carbamylation, carbonylation, biotinylation, carbamylation, oxidation, pegylation, sumoylation, ubiquitination, ubiquitylation, racemization, etc. One or more modifications may be made to the protein or peptide.

In some cases, the at least one therapeutic agent used to cause a difference in (e.g., a decrease or increase in) expression or activity of PKMYT1 may comprise a nucleic acid molecule, e.g., an RNA molecule. The RNA molecule can comprise any suitable RNA molecule and size sufficient to decrease the expression level or activity of PKMYT1, and, in some instances, PPP2R2A. The RNA molecule may comprise a small hairpin RNA (shRNA) molecule, a small interfering RNA (siRNA), a microRNA (miRNA), or other useful RNA molecule. In some examples, the RNA molecule may comprise a messenger RNA (mRNA), transfer RNA (tRNA), ribosomal RNAs (rRNA), small nuclear RNA (snRNA), piwi-interacting (piRNA), non-coding RNA (ncRNA), long non-coding RNA, (lncRNA), and fragments of any of the foregoing. The RNA molecule may be single-stranded, double-stranded, or partially single- or double-stranded.

It will be appreciated that the therapeutic agents (e.g., peptides, RNA molecules, protein-nucleic acid complexes) are listed as examples and that a combination of therapeutic agent types may be used to treat the subject. For instance, the composition may comprise one or more different types of therapeutic agents that may be used to decrease the expression or activity of PKMYT1. For example, a protein or peptide may be co-administered with a small molecule (e.g., a molecule having a molecular weight of less than 900 Daltons), an RNA molecule, a DNA molecule, or a complexed molecule (e.g., protein-nucleic acid molecule). Similarly, an RNA molecule may be administered with a small molecule, a DNA molecule, or a complexed molecule. In another example, a small molecule may be co-administered with a DNA molecule or a complexed molecule. These combinations are non-limiting examples of different combinations of agents that may be used to treat the subject having or suspected of having cancer (e.g., liver or ovarian cancer).

The composition may also comprise an excipient. The excipient may comprise a substance, which substance may be used to confer a property to the therapeutic agent or agents used to decrease the expression or activity level of PKMYT1. For instance, the excipient may comprise a substance for stabilization of the therapeutic agent. The excipient may comprise a substance for bulking up a solid, liquid, or gel formulation of the therapeutic agent. In some cases, the substance may confer a therapeutic enhancement to the therapeutic agent (e.g., by enhancing solubility). The substance may be used to change a property of the composition, such as the viscosity. The substance may be used to change a property of the therapeutic agent, e.g., bioavailability, absorption, hydrophilicity, hydrophobicity, pharmacokinetics, etc. The excipient may comprise a binding agent, anti-adherent agent, a coating, a disintegrant, a glidant (e.g., silica gel, talc, magnesium carbonate), a lubricant, a preservative, a sorbent, a sweetener, a vehicle, or a combination thereof. For instance, the excipient may comprise a powder, a mineral, a metal, a sugar (e.g. saccharide or polysaccharide), a sugar alcohol, a naturally occurring polymer (e.g., cellulose, methylcellulose) synthetic polymer (e.g., polyethylene glycol or polyvinylpyrrolidone), an alcohol, a thickening agent, a starch, a macromolecule (e.g., lipid, protein, carbohydrate, nucleic acid molecule), etc.

### Delivery or Administration of One or More Therapeutic Agents

The present disclosure provides methods and compositions for delivery, administration of, or exposure to one or more therapeutic agents described herein. One or more therapeutic agents may be delivered to a subject (e.g., in vivo), or to a cell or population of cells from a subject (e.g., ex vivo or in vivo). In some cases, the one or more therapeutic agents may be delivered to a subject in one or more delivery vesicles, such as a nanoparticle. The nanoparticle may be any suitable nanoparticle and may be a solid, semi-solid, semi-liquid or a gel. The nanoparticle may be a lipophilic and amphiphilic particle. For example, a nanoparticle may comprise a micelle, liposome, exosome, or other lipid-containing vesicle. In some cases, the nanoparticle may be configured for targeted delivery to a certain cell or cell type (e.g., cancer cell). In such cases, the nanoparticle may be decorated with any number of ligands, e.g., antibodies, nucleic acid molecules (e.g., ribonucleic acid (RNA) molecules or deoxyribonucleic acid (DNA) molecules), proteins, peptides, which may specifically bind to a certain cell or cell type (e.g., cancer cell).

The one or more therapeutic agents may be delivered using viral approaches. For example, the one or more therapeutic agents may be administered using a viral vector. In such cases, the one or more therapeutic agents may be encapsulated in a virus for delivery to a cell, population of cells, or the subject. The virus can be an adeno-associated virus (AAV), a retrovirus, a lentivirus, a herpes simplex virus, or other useful virus. The virus may be engineered or may be naturally occurring.

The one or more therapeutic agents may be delivered to a subject (e.g., human patient) or a body of the subject (e.g., at the tumor site) using a single or variety of approaches. For example, the one or more therapeutic agents may be delivered or administered orally, intravenously, intraperitoneally, intratumorally, subcutaneously, topically, transdermally, transmucosally, or through another administration approach.

The one or more therapeutic agents may be delivered to the subject enterally. For example, the one or more therapeutic agents may be administered to the subject orally, nasally, rectally, sublingually, sub-labially, buccally, topically, or through an enema. In such cases, the one or more therapeutic agents may be formulated into a tablet, capsule, drop or other formulation. The formulation may be configured to be delivered enterally.

The one or more therapeutic agents may be delivered to the subject parenterally. For example, the one or more therapeutic agents may be administered via injection into a location of the subject. The location may comprise the central nervous system, and the one or more therapeutic agents may be delivered epidurally, intracerebrally, intracerebroventricularly, etc. The location may comprise the skin, and the one or more therapeutic agents may be delivered epicutaneously. For instance, the one or more therapeutic agents may be formulated in a transdermal patch, which can deliver the one or more therapeutic agents to the skin of a subject. The one or more therapeutic agents may be delivered sublingually and/or bucally, extra-amniotically, nasally, intra-arterially, intra-articularly, intravavernously, intracardiacally, intradermally, intralesionally, intramuscularly, intraocularly, intraosseously, intraperitoneally, intrathecally, intrauterinely, intravaginally, intravenously, intravesically, intravitreally, subcutaneously, trans-dermally, perivascularly, transmucosally, or through another route of administration. In some cases, the one or more therapeutic agents may be delivered topically.

The one or more therapeutic agents may be formulated into an aerosol, pill, tablet, capsule (e.g., asymmetric membrane capsule), pastille, elixir, emulsion, powder, solution, suspension, tincture, liquid, gel, dry powder, vapor, droplet, ointment, patch, or a combination thereof. For instance, the one or more therapeutic agents may be formulated in a gel or polymer and delivered via a thin film.

In some instances, the one or more therapeutic agents may be delivered to the subject using a targeted delivery approach (e.g., for targeted delivery to the tumor site) or using a delivery approach to increase uptake of a cell of the one or more therapeutic agents. The delivery approach may comprise magnetic drug delivery (e.g., magnetic nanoparticle-based drug delivery), an acoustic targeted drug delivery approach, a self-microemulsifying drug delivery system, or other delivery approach. In some cases, the one or more therapeutic agents may be formulated for targeted delivery or for increased uptake of a cell. For example, the one or more therapeutic agents may be formulated with another agent, which may improve the solubility, hydrophobicity, hydrophilicity, absorbability, half-life, bioavailability, release profile, or other property of the one or more therapeutic agents. For example, the one or more therapeutic agents may be formulated with a polymer which may control the release profile of the one or more therapeutic agents. The one or more therapeutic agents may be formulated as a coating or with a coating (e.g., bovine submaxillary mucin coatings, polymer coatings, etc.) to alter a property of the one or more therapeutic agents (e.g., bioavailability, pharmacokinetics, etc.).

In some instances, the one or more therapeutic agents may be formulated using retrometabolic drug design. In such cases, the one or more therapeutic agents may be assessed for metabolic effects in a cell, and a new formulation comprising a derivative (e.g., chemically synthesized alternative or engineered variant) may be designed to change a property of the one or more therapeutic agents (e.g., to increase efficacy, minimize undesirable side effects, alter bioavailability, etc.).

### EXAMPLES

### Example 1—Identification of PKMYT1 and PPP2R2A as a Synthetic Lethal Pair

Valuable biomarkers in cancer cells can be mined from literature and public data, and further refinement of candidates can be performed using, for example, considerations such as multi-omics analysis, evaluation of tumor type (e.g. primary tumor), cell lines, target tractability, biomarker prevalence, etc. In one example of such screening, PPP2R2A and PKMYT1 may be identified as valuable biomarkers due to, for example, higher frequency of PPP2R2A in cancer cells compared to non-cancer cells and higher expression levels of PKMYT1 in cancer cells compared to non-cancer cells. Investigated cancer types may include, but are not limited to: acute myeloid leukemia (LAML), adrenocortical carcinoma (ACC), bladder urothelial carcinoma (BLCA), brain lower grade glioma (LGG), breast invasive carcinoma (BRCA), cervical squamous cell carcinoma and endocervical adenocarcinoma (CESC), cholangiocarcinoma (CHOL), chronic myelogenous leukemia (LCML), adenocarcinoma (COAD), esophageal carcinoma (ESCA), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), liver hepatocellular carcinoma (LIHC), lung adenocarcinoma (LUAD), lung squamous cell carcinoma (LUSC), lymphoid neoplasm diffuse large B-cell lymphoma (DLBC), mesothelioma (MESO), ovarian serous cystadenocarcinoma (OV), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), testicular germ cell tumors (TGCT), thymoma (THYM), thyroid carcinoma (THCA), uterine carcinosarcoma (UCS), uterine corpus endometrial carcinoma (UCEC), and uveal melanoma (UVM).

For instance, FIG. 2 shows a plot of frequency (Y-axis) of PPP2R2A inactivation or deficiency in different cancer types (X-axis). In certain types of cancers (e.g., prostate adenocarcinoma), the frequency of mutation of PPP2R2A can be as high as about 15%. In certain other cancer types (e.g., ovarian serous cystadenocarcinoma, rectum adenocarcinoma), the frequency of mutation of PPP2R2A may be greater than 10%. In various cancer types, the deficiency of PPP2R2A leading to inactivation may include: hypermethylation, deep deletion, or mutation in the PPP2R2A gene.

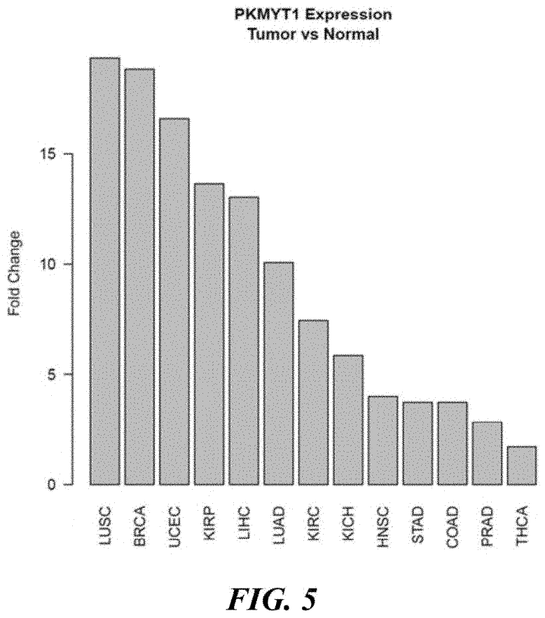

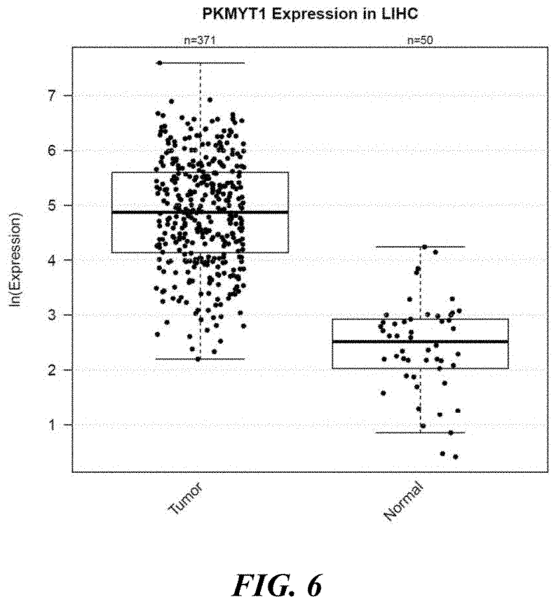

FIG. 5 shows a plot of the expression level of PKMYT1 in cancer versus normal (non-cancer) cells (Y-Axis, displayed as fold change) in various cancer types (X-Axis). In certain types of cancer (e.g., lung squamous cell carcinoma), the expression level of PKMYT1 is elevated compared to non-cancer cells. FIG. 6 shows a scatterplot of expression level (Y-Axis, displayed as ln(Expression)) of PKMYT1 in cancer (tumor) cells (n=371) compared to normal cells (n=50). As can be noted from the plot, PKMYT1 expression in cancer cells may be significantly higher than in normal cells. Altogether, FIGS. 5-6 demonstrate that PKMYT1 may be more highly expressed in various cancer types.

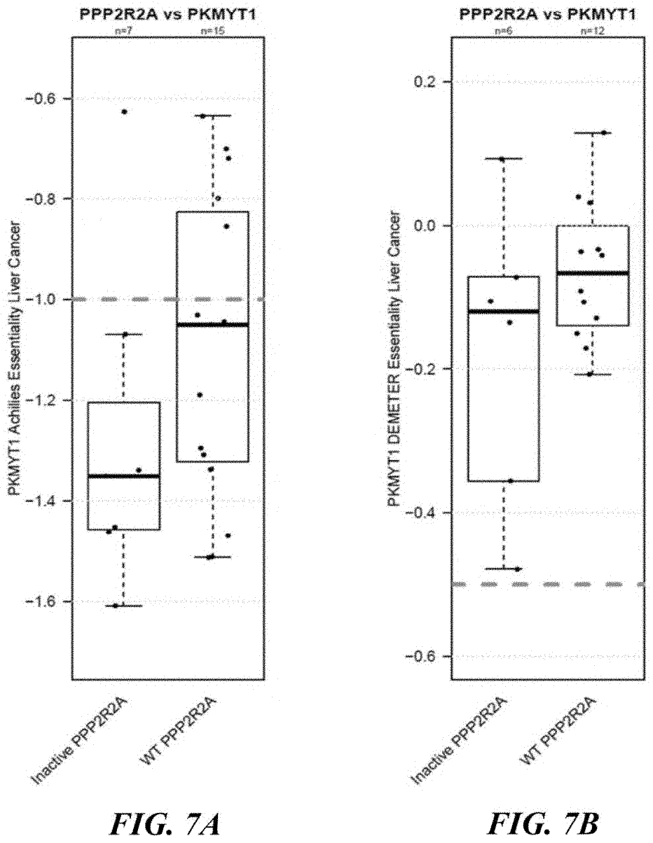

Further screening of PPP2R2A and PKMYT1 as a possible synthetic lethal pair may be performed. For instance, the expression level of PKMYT1 across a population of cells that have inactive or deficient PPP2R2A may be compared to the expression level of PKMYT1 across a population of cells that have a normal or wild-type genotype or phenotype of PPP2R2A. FIG. 7A shows a scatterplot of Achilles essentiality score (Y-Axis) of PKMYT1 in two populations of cells, either having the inactive or deficient PPP2R2A (e.g., mutated PPP2R2A) (n=7 cells) or having the wild-type PPP2R2A (n=15 cells). FIG. 7B shows a scatterplot of DEMETER essentiality score (Y-Axis) of PKMYT1 in two populations of cells, either having the inactive or deficient PPP2R2A (e.g., mutated PPP2R2A) (n=6 cells) or having the wild-type PPP2R2A (n=12 cells). In the population of cells having the PPP2R2A deficiency, PKMYT1 is more essential than for the population of cells having the wild-type PPP2R2A; that is, PKMYT1 knockdown is more lethal in the PPP2R2A-deficient cells than in the wild-type cells. These data may suggest that PPP2R2A and PKMYT1 may be a potential candidate of a synthetic lethal pair, and that knockdown of both genes may result in cell death, or that knockdown of PKMYT1 in PPP2R2A-deficient cells may result in cell death.

### Example 2—PKMYT1 and PPP2R2A as a Synthetic Lethal Pair

PKMYT1-PPP2R2A synthetic lethality may be tested experimentally. In one specific approach, the PKMYT1 and PPP2R2A genes may be knocked down or knocked out of a cell's genome using a combinatorial genetics CRISPR approach (e.g., combinatorial genetics en masse (CombiGEM)). In such an example, a DNA construct may be generated. The DNA construct may comprise a PKMYT1 gRNA to direct an endonuclease (e.g., a Cas protein) to the PKMYT1 gene, as well as a PPP2R2A gRNA to direct an endonuclease (e.g., a Cas protein) to the PPP2R2A gene. The PKMYT1 gRNA and PPP2R2A gRNA may comprise a sequence homologous or complementary to a sequence on the endogenous PKMYT1 gene and PPP2R2A gene, respectively. In some instances, the DNA construct may also comprise replacement genes to replace PKMYT1 and PPP2R2A in the genome (e.g., dysfunctional sequences, random DNA sequences).

Control DNA constructs may also be generated. For example, to determine if the gene pair is synthetic lethal, it may be important to monitor the effect of disrupting PKMYT1 and PPP2R2A individually as well as the combination of the gene pair. Moreover, it may be important to monitor the effect of a negative control, in which a DNA construct comprising an ineffective gRNA e.g., non-specific gRNA as a “non-cutting” control for one or both genes may be constructed. Another example of a negative control construct may comprise a vehicle control. In some cases, a positive control may also be used. The positive control may comprise, for instance, a DNA construct comprising a gRNA for a polymerase (e.g., an RNA polymerase, e.g., POLR2D), which can demonstrate that knockout (and the delivery mechanisms of doing so) of a gene that is essential for cell viability or proliferation results in lethality. In another example of a positive control, knockout of two genes known to be a synthetic lethal pair (e.g., methylthioadenosine phosphorylase (MTAP) and protein arginine methyltransferase 5 (PRMT5)) may be performed, e.g., using DNA constructs comprising gRNA directed to each of the known synthetic lethal genes.

The DNA constructs may then be introduced to cancer (e.g., liver or ovarian cancer) cells, which may comprise cells from a primary source (e.g., isolated from a tumor or cancer) or a cell line. An endonuclease, e.g., Cas9, may also be introduced to the cancer cells. The Cas9 may then replace, edit, or delete the PKMYT1 and PPP2R2A genes in the treated cells, and in some cases, replace the PKMYT1 and PPP2R2A genes in the genomes with the replacement genes in the DNA constructs. Proliferation or viability of the cells may then be monitored over time to determine the effectiveness of the treatment. The viability of the cells may be normalized or compared to a negative control or control population of cells that are not treated.

A sensitive florescence PrestoBlue assay based on production of resorufin (blue) from a substrate (colorless) by metabolically active cells was developed to quantify viable cells. Briefly, test plates were started with seeding 18,000 cells per well in a 96 well plate. The cells were transduced with a viral volume between 2-10 μL per well, depending on the viral titers obtained for achieving greater than 90% transduction. After 32 h post-transduction, media was changed to antibiotic (Puromycin) containing media and antibiotic was maintained for the rest of the assay. On Day 3 the plate was split and reseeded to an amount previously qualified to reach confluency in 14 days.

A total of 8 constructs were prepared for each gene pair tested: 2 sgRNA each for Gene A, paired with NTC (sg1, sg2), 2 sgRNA each for Gene B, paired with NTC (sg1, sg2), 4 sgRNA combinations (1,1; 1,2; 2,1; 2,2).

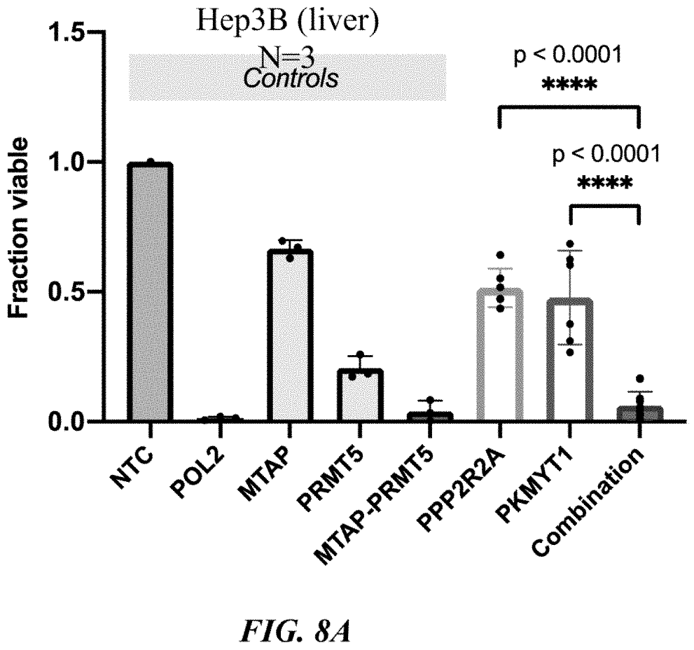

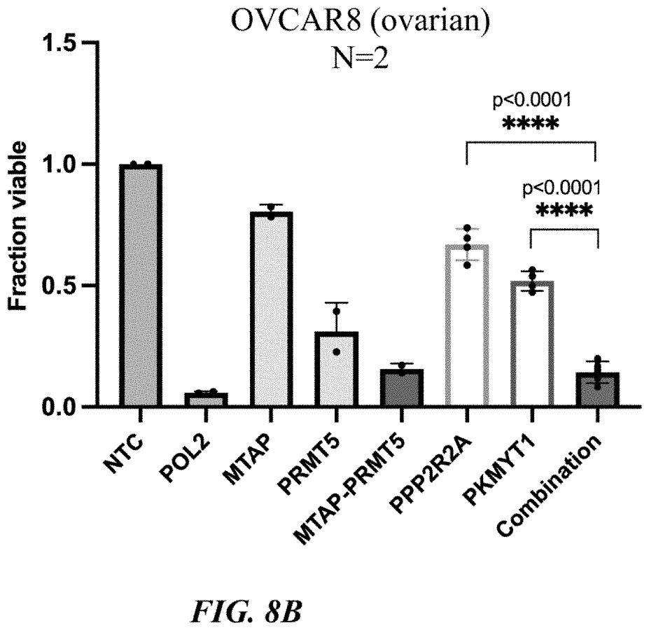

FIGS. 8A-B show example data of a CRISPR-based approach to knock out PKMYT1 and PPP2R2A. FIG. 8A illustrates bar plots of cell viability as a function of the DNA construct introduced. The DNA constructs can be used for knockout and can comprise: (i) a dual-negative control (NTC) sequence, (ii) a polymerase (POL2) sequence as a positive control for knockout of an essential gene, (iii) a MTAP sequence for knockout, (iv) a PRMT5 sequence for knockout, (v) MTAP and PRMT5 sequences for knockout, which can serve as a positive synthetic lethal control, (vi) PPP2R2A sequences for knockout, (vii) PKMYT1 sequences for knockout, and (viii) PPP2R2A and PKMYT1 sequences for dual knockout. The positive control sequence can be a DNA construct comprising a dysfunctional RNA polymerase gene (e.g., POLR2D gene) to replace the endogenous POLR2D gene, or the DNA construct may be configured to knock down or knock out a polymerase gene. The positive control sequence may be used, for example, to determine that the DNA constructs function as expected, e.g., that knock out of a gene essential for DNA replication, and thus cell proliferation, results in decreased cell viability.

The viability of the treated cells can be normalized to a negative control (e.g., non-treated cells, or cells treated with DNA constructs comprising scrambled gRNA or comprising normal copies of PKMYT1 and PPP2R2A). As can be seen in FIG. 8A, the negative control group of cells (NTC) has viability that is highest amongst the tested groups. The positive control (POL2), where the cells are treated with a DNA construct to knockout a polymerase, results in dramatically decreased normalized viability, as expected. The positive control (MTAP-PRMT5), where the cells are treated with a DNA construct to knockout MTAP and PRMT5, also results in decreased viability compared to the negative control groups. The cells that are treated with a single gene knockout, either PPP2R2A or PKMYT1, also show reduced viability compared to the negative control group. Knock out of PPP2R2A and PKMYT1 results in much lower viability than the single-knockout of PPP2R2A (p<0.0001) and the negative controls. Error bars represent standard deviation, n=3.

FIG. 8B illustrates another example of bar plots of cell viability as a function of the DNA construct introduced. The viability can be measured as a percentage of viable cells compared to a negative control. The DNA constructs can be used for knockout and can comprise: (i) a dual-negative control (NTC) sequence, (ii) a polymerase (POL2) sequence as a positive control for knockout of an essential gene, (iii) a MTAP sequence for knockout, (iv) a PRMT5 sequence for knockout, (v) MTAP and PRMT5 sequences for knockout, which can serve as a positive synthetic lethal control, (vi) a PPP2R2A sequences for knockout, (vii) a PKMYT1 sequences for knockout, and (viii) PPP2R2A and PKMYT1 sequences for dual knockout.

The viability of the treated cells can be normalized to a negative control (e.g., non-treated cells, or cells treated with DNA constructs comprising scrambled gRNA or comprising normal copies of PKMYT1 and PPP2R2A). Similar to FIG. 8A, the negative control group of cells (NTC) in FIG. 8B has viability that is highest amongst the tested groups. The positive control (POLR2D), where the cells are treated with a DNA construct to knockout a polymerase, results in dramatically decreased normalized viability, as expected. The positive control (MTAP-PRMT5), where the cells are treated with a DNA construct to knockout MTAP and PRMT5, also results in decreased viability compared to the negative control groups, as well as single gene knockouts of MTAP or PRMT5 alone. The cells that are treated with a single gene knockout, either PPP2R2A or PKMYT1, also show reduced viability compared to the negative control group. Knock out of PPP2R2A and PKMYT1 results in significantly lower viability than the single-knockout of PPP2R2A or the single-knockout of PKMYT1. Error bars represent standard deviation, n=2.

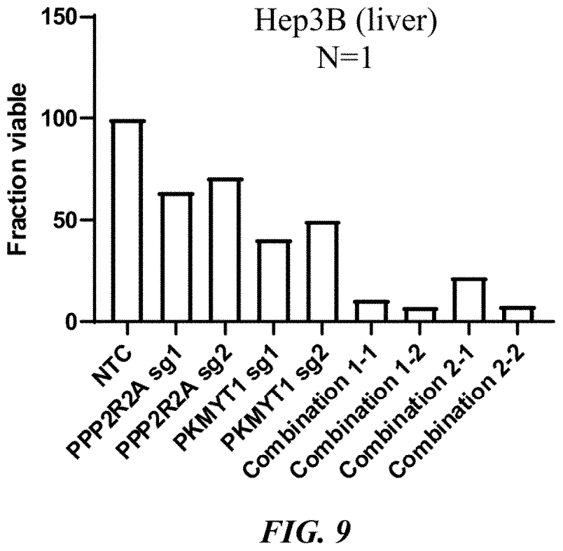

FIG. 9 illustrates another example of bar plots of cell viability as a function of the DNA construct introduced in a colony-forming assay (e.g.- clonogeneic assay). The viability can be measured as a percentage of viable cells compared to a negative control. The DNA constructs can be used for knockout and can comprise: (i) a negative control (NTC) sequence, (ii) a first sgRNA PPP2R2A sequence for knockout, (iii) a second sgRNA PPP2R2A sequence for knockout, (iv) a first sgRNA PKMYT1 sequence for knockout, (v) a second sgRNA PKMYT1 sequence for knockout, (vi) a first sgRNA PPP2R2A sequence and a first sgRNA PKMYT1 sequence for dual knockout, (vii) a first sgRNA PPP2R2A sequence and a second sgRNA PKMYT1 sequence for dual knockout, (viii) a second sgRNA PPP2R2A sequence and a first sgRNA PKMYT1 sequence for dual knockout, and (ix) a second sgRNA PPP2R2A sequence and a second sgRNA PKMYT1 sequence for dual knockout.

The viability of the treated cells can be normalized to a negative control (e.g., non-treated cells, or cells treated with DNA constructs comprising scrambled gRNA or comprising normal copies of PKMYT1 and PPP2R2A). Similar to FIGS. 8A-B, the negative control group of cells (NTC) in FIG. 9 has viability that is highest amongst the tested groups. The cells that are treated with a single gene knockout, either PPP2R2A or PKMYT1, show reduced viability compared to the negative control group. Knock out of PPP2R2A and PKMYT1 results in significantly lower viability than the single-knockout of PPP2R2A or the single-knockout of PKMYT1.

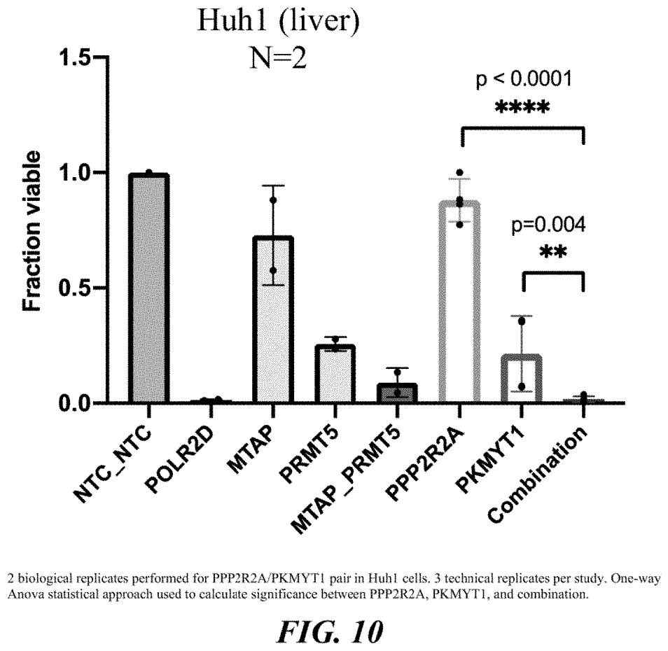

Huh1 has a homozygous deletion of PPP2R2A. PKMYT1 deletion is expected to be lethal irrespective of PPP2R2A CRISPR KO. As predicted, PKMYT1 knockout alone shows strong cell killing in Huh1 cells which have an endogenous deletion of the PPP2R2A gene locus (FIG. 10). The strength of the synthetic lethal interaction of PPP2R2A-PKMYT1 is summarized for 4 different cell lines, including the colorectal cancer cell line HCT116, in Table 1.

TABLE 1
PPP2R2A-PKMYT1 in 4 Cell Lines

| SL | | | | Fractional |
|---|---|---|---|---|
| categorization* | Cell Line | Indication | EOB** | Viability |
| SL | Hep3B | HCC | 0.52 | 0.17 |
| SL | OVCAR8 | Ovarian | 0.51 | 0.21 |
| SL | HCT116 | CRC | 0.03 | 0.06 |
| SL | Huh1 | HCC | 0.17 | 0.01 |

*Synthetic Lethal (SL) has fractional viability (FV) of gene combination < 0.2 (20%). Synthetic Sick (SS) has fractional viability of gene combination of 0.2 to 0.5 (20%-50%). No Effect (NE) has fractional viability of gene combination of 0.5 to 1.0 (50%-100%).
**EOB = (FVgeneA × FVgeneB) − (FVgeneAB).

There is low residual expression of PPP2R2A in Huh1 as shown in Table 2. CRISPR knockout of both PKMYT1 and PPP2R2A in Huh1 eliminates the residual expression of PPP2R2A in this cell line and causes a further reduction in cell viability.

TABLE 2
PPP2R2A Expression in Cell Lines

| | | PPP2R2A | |
|---|---|---|---|
| Cas9 cell | Treatment | Relative | |
| line | (sample) | Quantity* | Explanation |
| Hep3B | None | 100 | Control, maximum expression |
| | | | of PPP2R2A |
| Hep3B | PPP2R2Asg1, | 1.3 | CRISPR Knockout of PPP2R2A |
| | Day 14 | | reduces gene expression |
| Huh1 | None | 6.7 | Reduced expression in Huh1 |
| OVCAR8 | None | 11.2 | Reduced expression in |
| | | | OVCAR8 |

*Expression levels of PPP2R2A were determined using antibodies directed against the PPP2R2A protein in cell lines under different treatment conditions as noted.

There is high expression of PKMYT1 in Huh1 as shown in Table 3. CRISPR knockout of both PKMYT1 and PPP2R2A in Huh1 eliminates the expression of PKMYT1 in this cell line and causes a further reduction in cell viability.

TABLE 3
PKMYT1 Expression in Cell Lines

| | | PKMYT1 | |
|---|---|---|---|
| Cas9 cell | Treatment | Relative | |
| line | (sample) | Quantity | Explanation |
| Hep3B | None | 100 | Basal expression of PKMYT1 in |
| | | | Hep3B |
| Hep3B | PKMYT1sg1, | 36 | CRISPR Knockout of PKMYT1 |
| | Day 14 | | reduces gene expression |
| Huh1 | None | 166 | Increased expression in Huh1 vs |
| | | | Hep3B |
| OVCAR8 | None | 72 | Reduced expression in |
| | | | OVCAR8 vs Hep3B |

*Expression levels of PMKYT1 were determined using antibodies directed against the PKMYT1 protein in cell lines under different treatment conditions as noted.

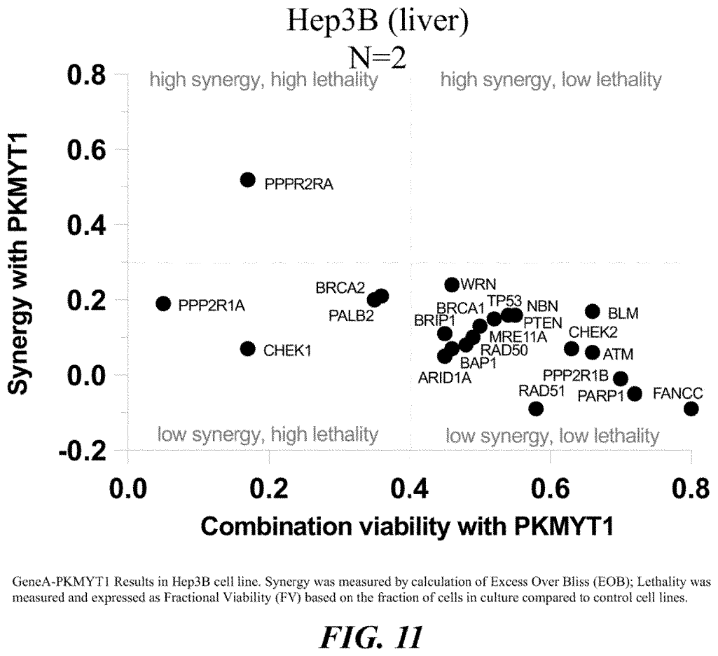

A selection of genes involved in DNA repair were identified by systems biology analysis as a potential pathway explaining the Synthetic Lethality of PPP2R2A with PKMYT1. A selection of 22 different genes known to be involved in DNA repair by the Homology Directed Repair mechanism (HDR) were screened to determine their interaction with PKMYT1. The results of this screen are summarized in Table 4 and FIG. 11. These screens showed that only PPP2R2A had high synergy (excess over Bliss “EOB”) and strong cell killing (Fractional Viability, FV) with PKMYT1. This uniquely strong interaction between PPP2R2A and PKMYT1 had not been reported in the literature and was an unexpected observation.

TABLE 4
Synthetic Lethality Screen Results

| | | Fractional | SL |
|---|---|---|---|
| Gene Pair* | EOB | Viability | categorization |
| PPPR2R2A-PKMYT1 | 0.52 | 0.17 | SL |
| PPP2R1A-PKMYT1 | 0.19 | 0.05 | SL |
| CHEK1-PKMYT1 | 0.07 | 0.17 | SL |
| PALB2-PKMYT1 | 0.20 | 0.35 | SS |
| BRCA2-PKMYT1 | 0.21 | 0.36 | SS |
| BRIP1-PKMYT1 | 0.11 | 0.45 | SS |
| ARID1A-PKMYT1 | 0.05 | 0.45 | SS |
| BAP1-PKMYT1 | 0.07 | 0.46 | SS |
| WRN-PKMYT1 | 0.24 | 0.46 | SS |
| RAD50-PKMYT1 | 0.08 | 0.48 | SS |
| MRE11A-PKMYT1 | 0.10 | 0.49 | SS |
| PTEN-PKMYT1 | 0.13 | 0.50 | SS |
| BRCA1-PKMYT1 | 0.15 | 0.52 | NE |
| NBN-PKMYT1 | 0.16 | 0.54 | NE |
| TP53-PKMYT1 | 0.16 | 0.55 | NE |
| RAD51-PKMYT1 | −0.09 | 0.58 | NE |
| CHEK2-PKMYT1 | 0.07 | 0.63 | NE |
| BLM-PKMYT1 | 0.17 | 0.66 | NE |
| ATM-PKMYT1 | 0.06 | 0.66 | NE |
| PPP2R1B-PKMYT1 | −0.01 | 0.7 | NE |
| PARP1-PKMYT1 | −0.05 | 0.72 | NE |
| FANCC-PKMYT1 | −0.09 | 0.80 | NE |
| ATRX-PKMYT1 | −0.02 | 0.88 | NE |

A selection of 22 different genes known to be involved in DNA repair by the Homology Directed Repair mechanism (HDR) were screened in Hep3B cells to determine their interaction with PKMYT1. These screens showed that only PPP2R2A had high synergy (EOB) and strong cell killing as Fractional Viability (FV).

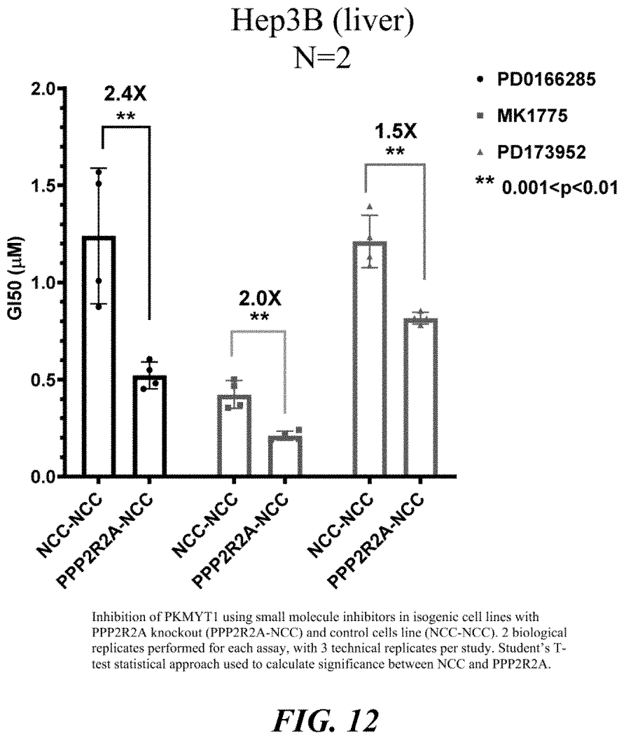

Inhibition of PKMYT1 by small molecule inhibitors can replicate the findings of CRISPR-mediated knockout of PKMYT1. PPP2R2A expression is reduced using CRISPR knockout and small molecule drugs are subsequently applied to the cells (Table 5, FIG. 12). Control cells are also used where PPP2R2A is not knocked out. It is observed that when PPP2R2A is knocked out in a cell line the PKMYT1 inhibitors show increased potency.

TABLE 5
Small Molecule Inhibitors

| Inhibitor | PKMYT1 IC₅₀ (nM) | Wee1 IC₅₀ (nM) |
|---|---|---|
| PD0166285 | 8.2 | 0.85 |
| MK1775 | 1,100 | 2.4 |
| PD173952 | 46 | 5.3 |

The in vitro potency of the three compounds tested in the PPP2R2A and control cell lines was assessed by selective binding assays for PKMYT1 and Wee1.

Altogether, these results support that PPP2R2A and PKMYT1 may be a synthetic lethal pair. In such cases, treatment of PPP2R2A-deficient cells with a therapeutically effective amount of one or more therapeutic agents that cause decreased activity level or expression of PKMYT1 may be a viable treatment option.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It will be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-58. (canceled)

59. A method for treating a subject having or suspected of having a cancer, comprising:
administering to said subject a therapeutically effective amount of a therapeutic agent, wherein the administration results in a difference in expression or activity of protein kinase, membrane associated tyrosine/threonine 1 (PKMYT1) or WEE1 G2 checkpoint kinase (WEE1) in said subject; and
wherein said cancer comprises a cell comprising a difference in expression or activity of Protein Phosphatase 2 (PP2A) or a PP2A subunit, or comprising a mutation or deletion of a nucleic acid encoding the PP2A subunit, as compared to a non-cancer control.

60. The method of claim 59, wherein said cell comprises the difference in expression or activity of PPA2 or the PP2A subunit.

61. The method of claim 60, wherein said difference in expression or activity of PP2A or the subunit thereof comprises a decrease in the expression or activity.

62. The method of claim 59, wherein said cell comprises the mutation or deletion of the nucleic acid encoding the PP2A subunit.

63. The method of claim 59, further comprising identifying the cancer as comprising the cell comprising the difference in expression or activity of PP2A or the PP2A subunit, or as comprising the mutation or deletion of the nucleic acid encoding the PP2A subunit.

64. The method of claim 59, wherein said therapeutic agent comprises a small molecule, a protein, or a nucleic acid.

65. The method of claim 64, wherein said therapeutic agent comprises a small molecule.

66. The method of claim 65, wherein said small molecule comprises a PKMYT1 inhibitor.

67. The method of claim 66, wherein said PKMYT1 inhibitor comprises 5-((5-methoxy-2-((4-morpholinophenyl)amino)pyrimidin-4-yl)amino)-2-methylphenol, N-(2-chloro-6-methylphenyl)-2-((6-(4-(2-hydroxyethyl)piperazin-1-yl)-2-methylpyrimidin-4-yl)amino)thiazole-5-carboxamide (dasatinib), 4-((2,4-dichloro-5-methoxyphenyl)amino)-6-methoxy-7-(3-(4-methylpiperazin-1-yl)propoxy)quinoline-3-carbonitrile (bosutinib), N-(5-chlorobenzo[d][1,3]dioxol-4-yl)-7-(2-(4-methylpiperazin-1-yl)ethoxy)-5-((tetrahydro-2H-pyran-4-yl)oxy)quinazolin-4-amine (saracatinib), (E)-N-(4-((3-chloro-4-fluorophenyl)amino)-3-cyano-7-ethoxyquinolin-6-yl)-4-(dimethylamino)but-2-enamide (pelitinib), N-(3-chlorophenyl)-6,7-dimethoxyquinazolin-4-amine (tyrphostin AG 1478), 6-(2,6-dichlorophenyl)-2-((4-(2-(diethylamino)ethoxy)phenyl)amino)-8-methylpyrido[2,3-d]pyrimidin-7(8H)-one (PD-0166285), 6-(2,6-dichlorophenyl)-8-methyl-2-((4-morpholinophenyl)amino)pyrido[2,3-d]pyrimidin-7(8H)-one (PD-173952), 6-(2,6-dichlorophenyl)-8-methyl-2-((3-(methylthio)phenyl)amino)pyrido[2,3-d]pyrimidin-7(8H)-one (PD-173955), or 6-(2,6-dichlorophenyl)-2-((4-fluoro-3-methylphenyl)amino)-8-methylpyrido[2,3-d]pyrimidin-7(8H)-one (PD-180970).

68. The method of claim 65, wherein said small molecule comprises a WEE1 inhibitor.

69. The method of claim 68, wherein said WEE1 inhibitor comprises 6-(2,6-dichlorophenyl)-2-((4-(2-(diethylamino)ethoxy)phenyl)amino)-8-methylpyrido[2,3-d]pyrimidin-7(8H)-one (PD-0166285), 2-allyl-1-(6-(2-hydroxypropan-2-yl)pyridin-2-yl)-6-((4-(4-methylpiperazin-1-yl)phenyl)amino)-1,2-dihydro-3H-pyrazolo[3,4-d]pyrimidin-3-one (MK-1775), 9-hydroxy-4-phenylpyrrolo[3,4-c]carbazole-1,3(2H,6H)-dione (PD-407824), 6-butyl-4-(2-chlorophenyl)-9-hydroxypyrrolo[3,4-c]carbazole-1,3(2H,6H)-dione, or 6-(2-chloro-6-fluorophenyl)-2-((2,4,4-trimethyl-1,2,3,4-tetrahydroisoquinolin-7-yl)amino)imidazo[1,2-a]pyrimido[5,4-e]pyrimidin-5(6H)-one.

70. The method of claim 59, wherein said cell comprises the difference in expression or activity of the PP2A subunit, or comprises the mutation or deletion of the nucleic acid encoding the PP2A subunit; and
wherein said PP2A subunit comprises 65 kDa regulatory subunit A alpha (PPP2R1A), 65 kDa regulatory subunit A beta (PPP2R1B), 55 kDa regulatory subunit B alpha (PPP2R2A), 55 kDa regulatory subunit B beta (PPP2R2B), 55 kDa regulatory subunit B gamma (PPP2R2C), 55 kDa regulatory subunit B delta (PPP2R2D), 72/130 kDa regulatory subunit B (PPP2R3A), 48 kDa regulatory subunit B (PPP2R3B), regulatory subunit B″ subunit gamma (PPP2R3C), regulatory subunit B′ (PPP2R4), 56 kDa regulatory subunit alpha (PPP2R5A), 56 kDa regulatory subunit beta (PPP2R5B), 56 kDa regulatory subunit gamma (PPP2R5C), 56 kDa regulatory subunit delta (PPP2R5D), 56 kDa regulatory subunit epsilon (PPP2R5E), catalytic subunit alpha (PPP2CA), or catalytic subunit beta (PPP2CB).

71. The method of claim 70, wherein said PP2A subunit comprises PPP2R1A.

72. The method of claim 70, wherein said PP2A subunit comprises PPP2R2A.

73. The method of claim 59, further comprising administering to said subject a therapeutically effective amount of a second therapeutic agent comprising an anti-cancer agent.

74. The method of claim 59, wherein said cancer comprises a cancerous tissue comprising said cell.

75. The method of claim 74, wherein said cancerous tissue comprises breast tissue, pancreatic tissue, uterine tissue, bladder tissue, colorectal tissue, prostate tissue, liver tissue, or ovarian tissue.

76. The method of claim 74, wherein said cancerous tissue is liver tissue.

77. The method of claim 74, wherein said cancerous tissue is ovarian tissue.

78. The method of claim 59, wherein the subject has the cancer, and the administration reduces proliferation or viability of the cancer.
